(19) United States
(12) Patent Application Publication
(10) Pub. No.: US 2015/0203546 A1
(43) Pub. Date: Jul. 23, 2015

(54) SCYTODES VENOM FIBER PEPTIDES, NUCLEIC ACIDS AND METHODS OF MAKING AND USING

(71) Applicant: PAMELA A. ZOBEL-THROPP, Lake Oswego, OR (US)

(72) Inventor: PAMELA A. ZOBEL-THROPP, Lake Oswego, OR (US)

(21) Appl. No.: 14/602,995

(22) Filed: Jan. 22, 2015

Related U.S. Application Data
(60) Provisional application No. 61/930,309, filed on Jan. 22, 2014.
(60) Provisional application No. 61/930,322, filed on Jan. 22, 2014.
(60) Provisional application No. 61/930,742, filed on Jan. 23, 2014.
(60) Provisional application No. 61/930,786, filed on Jan. 23, 2014.

(51) Int. Cl. C07K 14/435

(52) U.S. Cl. CPC C07K 14/43518; C07K 2319/35; C07K 2319/034; C07K 2319/21; C07K 2319/24

(57) ABSTRACT

The present invention is directed to spider silk-like fibers, peptides comprising the fibers, nucleic acids encoding the peptides, nucleic acid constructs and recombinant expression vectors, fusion peptides and methods of making and using the foregoing.

### CROSS-REFERENCE TO RELATED APPLICATIONS

This application is based on and claims priority to U.S. Provisional Application Ser. No. 61/930,309, filed on Jan. 22, 2014, Ser. No. 61/930,322, filed on Jan. 22, 2014, Ser. No. 61/930,742, filed on Jan. 23, 2014, and Ser. No. 61/930,786, filed on Jan. 23, 2014, which are incorporated herein by reference in their entirety.

### STATEMENT REGARDING FEDERALLY SPONSORED RESEARCH OR DEVELOPMENT

The present invention was supported in part by NSF Grant No. R15-GM-097696-01, and the government may have certain rights in the invention.

### INCORPORATION OF SEQUENCE LISTING

A paper copy of the Sequence Listing and a computer readable form of the Sequence Listing containing the file named “3000281-0003_ST25.txt”, which is 127,360 bytes in size (as measured in MICROSOFT WINDOWS® EXPLORER), are provided herein and are herein incorporated by reference. This Sequence Listing consists of SEQ ID NOs:1-276.

### BACKGROUND OF THE INVENTION

1. Field of the Invention

The present invention is generally related to the nonconventional spider silk-like fibers exuded from spider spit venom, peptides comprising the fibers, nucleic acids encoding the peptides and methods of making and using the foregoing.

2. Description of Related Art

Spiders are highly successful predators that use various techniques to capture prey. In conjunction with venom, many utilize multiple and varied forms of silk to ensnare, wrap, detect and protect themselves for survival. Several types of spider silks have been identified and analyzed because of their many possibilities for human use. Silk proteins are incredibly strong, elastic, and some are even sticky. They are resistant to heat and chemicals, lightweight, antimicrobial, hypoallergenic, and biodegradable (Römer and Scheibel, 2008). Scientists have applied these characteristics to strengthening armor and car doors, medical applications, such as internal stitches, capsules for drug delivery, nerve repair, and ligament replacement, and also making a new type of natural clothing. However, there is a significant challenge in mass production of spider silk proteins. Silk spidroin molecules are very large (>250 kDa) and consequently difficult to express in vitro; to date only segments of silk proteins have been expressed and purified. There has been some success in making artificial silk for commercial use, but none in expressing native, palpable silk as a recombinant protein.

The biochemical structure of these fibers is what defines their roles in nature, and also what makes them so difficult to create in vitro. These very long polypeptides contain multiple iterations of amino acid motifs (e.g. GPGxx, GGx (where “x” is any amino acid), poly-A, and/or poly-AG) interspersed with non-conserved stretches of amino acids. When folding into its correct conformation, the non-conserved regions serve as turning points so the polypeptide can fold back on itself allowing the side chains of the motif repeats to align, forming hydrogen bonds; as more and more folds stack, thick β-sheets form which comprise very strong structures (Kluge et al., 2008). These motifs are highly conserved and the side chain bonding properties have been characterized in many species (Gosline et al., 1999, 2002; Swanson et al., 2006; Stark et al., 2007; Savage and Gosline, 2008; Boutry and Blackledge, 2010; Perry et al., 2010; Prosdocimi et al., 2011; Sahni et al., 2011; Vasanthavada et al., 2012). More recent reports of novel silk and silk-like proteins identify glycine-rich motifs in sticky proteins (Maruyama et al. 2010), and motifs including diglutamines (QQ) and dityrosines (YY) that cross-link through their side chain residues (Shewry et al 2002; Feeney et al, 2003; Ayoub et al, 2007; Perry et al, 2010; Vasanthavada et al. 2012).

Of the >45,000 species of spiders identified to date (World Spider Catalog, 2015), Scytodes is the only spider that spits a combination of venom and sticky fiber to tether its prey before paralyzing and then killing with a toxic venom bite instead of expelling and using silk from their abdomen. Scytodes spiders have a domed cephalothorax, housing a pair of glands that produce both toxic venom and sticky fibers that are extruded through the same duct (Foelix, 1996). They have diminutive chelicerae and fangs, from which a viscous, gluey substance is rapidly sprayed independently from each fang in a zig-zag fashion. The substance is adhesive, contractile, and immobilizes prey while tethering them to a surface (Suter and Stratton, 2005, 2009). Suter and Stratton (2009) used high resolution microscopy to show that the spit contains a long, continuous fibrous strand connecting sticky glue droplets. These strands shrink 40-60% upon ejection from the fangs (Suter and Stratton, 2009), a major factor in capturing larger prey such as other spiders. Interestingly, direct application of the spit mixture on prey has no toxic effects (Clements and Li, 2005).

### BRIEF SUMMARY OF THE INVENTION

The present invention is directed to spider silk-like fibers, peptides comprising the fibers, nucleic acids encoding the peptides, nucleic acid constructs and recombinant expression vectors, fusion peptides and methods of making and using the foregoing.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 depicts an SDS-PAGE analysis of crude Scytodes spit (left lane). The circled region highlights the fiber, which did not dissolve or enter the resolving gel. The numbers correspond to molecular weight standards (right lane).

FIG. 2 depicts an alignment of selected peptides identified in a Scytodes venom gland transcriptome.

FIG. 3 depicts an SDS-PAGE analysis of a purified peptide of the present invention expressed in accordance with the present invention. The numbers correspond to molecular weight standards (outside lanes).

### DETAILED DESCRIPTION OF PREFERRED EMBODIMENT

The present invention is directed to spider silk-like fibers isolated from the spit of Scytodes spiders, peptides comprising the fibers, nucleic acids encoding the peptides, nucleic acid constructs and recombinant expression vectors, fusion peptides, and methods of making and using the foregoing.

Definitions:

The terms “peptide,” “oligopeptide,” “polypeptide,” “polyprotein,” and “protein,” are used interchangeably herein, and refer to a polymeric form of amino acids of any length, which can include coded and non-coded amino acids, chemically or biochemically modified or derivatized amino acids, and polypeptides having modified peptide backbones.

The term “recombinant,” as used herein with respect to DNA, means that a particular DNA sequence is the product of various combinations of cloning, restriction, and/or ligation steps resulting in a construct having a structural coding sequence distinguishable from homologous sequences found in natural systems. Generally, DNA sequences encoding the structural coding sequence can be assembled from cDNA fragments and short oligonucleotide linkers, or from a series of oligonucleotides, to provide a synthetic gene that is capable of being expressed in a recombinant transcriptional unit. Such sequences can be provided in the form of an open reading frame uninterrupted by internal nontranslated sequences, or introns, which are typically present in eukaryotic genes. Conversely, for stabilization purposes such sequences can be provided in the form of an open reading frame interrupted by insertion of artificial non-translated sequences, or introns, which naturally are not present in viral genes. Genomic DNA comprising the relevant sequences could also be used. Sequences of non-translated DNA, other than introns, may also be present 5′ or 3′ from the open reading frame, where such sequences do not interfere with manipulation or expression of the coding regions. Thus, for example, the term “recombinant” polynucleotide or nucleic acid refers to one which is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

The term “recombinant DNA” molecule means a hybrid DNA sequence comprising at least two nucleotide sequences not normally found together in nature.

The term “construct” generally refers to recombinant nucleic acid, generally recombinant DNA, that has been generated for the purpose of the expression of a specific nucleotide sequence(s), or is to be used in the construction of other recombinant nucleotide sequences.

Similarly, the terms “recombinant polypeptide” or “recombinant polyprotein” refers to a polypeptide or polyprotein that is not naturally occurring. For example, it may be expressed in a different organism than the one from which the encoding gene originated, or it may be made by the artificial combination of two otherwise separated segments of amino acid sequences. This artificial combination may be accomplished by standard techniques of recombinant DNA technology, such as described above, i.e., a recombinant polypeptide or recombinant polyprotein may be encoded by a recombinant polynucleotide. Thus, a recombinant polypeptide or recombinant polyprotein is an amino acid sequence encoded by all or a portion of a recombinant polynucleotide. In contrast, the term “native protein” is used herein to indicate a protein isolated from a naturally occurring (i.e., a nonrecombinant) source. Molecular biological techniques may be used to produce a recombinant form of a protein with identical properties as compared to the native form of the protein.

The term “fusion protein” refers to a recombinant polypeptide or recombinant polyprotein made by the artificial combination of two naturally separated segments of amino acid sequences.

The terms “mature peptide,” “mature protein” or “mature peptide sequence” refer to a peptide, protein or peptide sequence after removal of any native signaling peptides and other N terminal modifications.

The term “mature cDNA” means a cDNA that encodes a mature peptide.

The term “gene” refers to a DNA sequence that comprises coding sequences and optionally control sequences necessary for the production of a polypeptide from the DNA sequence.

The term “vector” is used in reference to nucleic acid molecules into which fragments of DNA may be inserted or cloned and can be used to transfer DNA segments into a cell and capable of replication in a cell. Vectors may be derived from plasmids, bacteriophages, viruses, cosmids, and the like.

The terms “recombinant vector” or “expression vector” as used herein refer to DNA or RNA sequences containing a desired coding sequence and appropriate DNA or RNA sequences necessary for the expression of the operably linked coding sequence in a particular host organism. Prokaryotic expression vectors include a promoter, a ribosome binding site, an origin of replication for autonomous replication in a host cell and possibly other sequences, e.g. an optional operator sequence, optional restriction enzyme sites. A promoter is defined as a DNA sequence that directs RNA polymerase to bind to DNA and to initiate RNA synthesis. Eukaryotic expression vectors include a promoter, optionally a polyadenylation signal and optionally an enhancer sequence.

A polynucleotide having a nucleotide sequence “encoding a peptide, protein or polypeptide” means a nucleic acid sequence comprising a coding region for the peptide, protein or polypeptide. The coding region may be present in cDNA, genomic DNA or RNA form. When present in a DNA form, the oligonucleotide may be single-stranded (i.e., the sense strand) or double-stranded. Suitable control elements such as enhancers/promoters, splice junctions, polyadenylation signals, etc. may be placed in close proximity to the coding region, of the gene if needed to permit proper initiation of transcription and/or correct processing of the primary RNA transcript. Alternatively, the coding region, utilized in the expression vectors of the present invention may contain endogenous enhancers/promoters, splice junctions, intervening sequences, polyadenylation signals, etc. In further embodiments, the coding region may contain a combination of both endogenous and exogenous control elements.

Promoters and enhancers consist of short arrays of DNA sequences that interact specifically with cellular proteins involved in transcription. Promoter and enhancer elements have been isolated from a variety of eukaryotic sources including genes in yeast, insect and mammalian cells. Promoter and enhancer elements have also been isolated from viruses and analogous control elements, such as promoters, are also found in prokaryotes. The selection of a particular promoter and enhancer depends on the cell type used to express the protein of interest. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques) such that transcription of the gene is directed by the linked enhancer/promoter. A “constitutive promoter” is an unregulated promoter that allows for continual transcription of its associated gene.

The term “expression system” refers to any assay or system for determining (e.g., detecting) the expression of a gene of interest. Those skilled in the field of molecular biology will understand that any of a wide variety of expression systems may be used.

The terms “cell,” “cell line,” “host cell,” as used herein, are used interchangeably, and all such designations include progeny or potential progeny of these designations. By “transformed cell” is meant a cell into which (or into an ancestor of which) has been introduced a nucleic acid molecule of the invention. Optionally, a nucleic acid molecule of the invention may be introduced into a suitable cell line so as to create a stably transfected cell line capable of producing the protein or polypeptide encoded by the nucleic acid molecule. Vectors, cells, and methods for constructing such cell lines are well known in the art. The words “transformants” or “transformed cells” include the primary transformed cells derived from the originally transformed cell without regard to the number of transfers. All progeny may not be precisely identical in DNA content, due to deliberate or inadvertent mutations. Nonetheless, mutant progeny that have the same functionality as screened for in the originally transformed cell are included in the definition of transformants.

The term “operably linked” as used, herein refers to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein of polypeptide, or a precursor thereof, e.g., the pre-or prepro-form of the protein or polypeptide, is produced.

Turning to the present invention, a number of unique cDNA sequences encoding glycine-rich peptides were identified in the venome (transcriptome and proteome components) of Scytodes thoracica. It was discovered that a majority of the cDNA sequences that make up all of the transcribed genes in the venom gland tissue are glycine-rich peptides, which constitute a novel gene family not found in any other organism. The mature sequences are short (˜42 aa after signal sequence processing), and contain GGx, QQ and YY motifs as in known silk and silk-like proteins. However, unlike known silk and silk-like proteins, the glycine-rich cDNA sequences identified in the venome of Scytodes thoracica are not extensively long or sequentially repetitive.

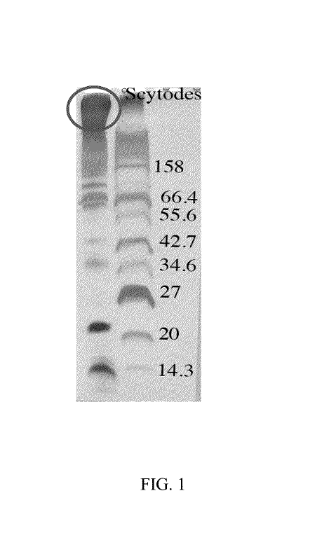

The high abundance of these unique transcripts, along with their silk-like motifs, suggests that these peptides are the adhesive and contractile strand components of the silk-like fibers in the spit. When venom is collected, the spit comes out like cotton candy that can be wound around a capillary tube. It is thick, viscous, clear, and does not dissolve in a buffer normally used to dissolve and analyze venom, even after two and up to four days of incubation in the same buffer. FIG. 1 depicts a visualization of the crude Scytodes spit (left lane) using SDS-PAGE (10-20% gel). The molecular weight standards are on the right. There is an obvious aggregate at the top of the left lane that did not enter the stacking gel or the resolving gel, showing its resistance to denaturing agents (SDS-PAGE loading buffer, a denaturant) and heat (boiling samples for 5 min just prior to loading), which is thought to be the silk-like fiber component. Proteomics analysis was done at the University of Arizona Proteomics Consortium via two methods: MudPIT and Orbitrap. Both methods utilize digested protein mixtures for mass spectrometry analysis, neither of which detected these glycine-rich proteins in abundance as expected. A request for the Proteomics facility to specifically cut out that aggregated band, digest it and run an additional analysis yielded no new results, which indicates trypsin-resistance as well.

SEQ ID NOs: 1-64 and 257-260 are cDNAs identified as genes encoding peptides having characteristics considered sufficient to be characterized as having potential function in the contraction and adhesion properties of the silk and silk-like peptides found in Scytodes spit, as described in Zobel-Thropp, P. A., Correa, S. M., Garb, J. E. and Binford, G. J. (2014) Spit and venom from Scytodes spiders: a diverse and distinct cocktail J. Proteome Res. 13: 817-835 DOI: 10.1021/pr400875s., which is incorporated herein by reference. These sequences are from a single gene family and have been identified to encode the contractile and adhesive fiber component that is spit from the spider's fangs during prey capture.

SEQ ID NOs: 65-128 and 261-264 were identified as the full length peptides that are encoded by such cDNAs. These peptides contain a signal peptide sequence which suggests that the peptides are processed for secretion. SEQ ID NOs: 129-192 and 265-268 are the mature cDNA sequences. SEQ ID NOs: 193-256 and 269-272 are the mature peptide sequences after cleavage of the signal peptide sequences. The peptides are small, less than 100 amino acids, and in fact less than 75 amino acids and less than 50 amino acids.

The glycine-rich gene and peptide families were phylogenetically resolved into 6 distinct clades, as follows:

TABLE 1
Clades

| Clade | Full length cDNA | Full length amino acid | Mature cDNA | Mature amino acid |
|---|---|---|---|---|
| I | SEQ ID NOs: 1-7 | SEQ ID NOs: 65-71 | SEQ ID NOs: 129-135 | SEQ ID NOs: 193-199 |
| II | SEQ ID NOs: 8-38, 260 | SEQ ID NOs: 72-102, 264 | SEQ ID NOs: 136-166, 268 | SEQ ID NOs: 200-230, 272 |
| III | SEQ ID NOs: 39-47 | SEQ ID NOs: 103-111 | SEQ ID NOs: 167-175 | SEQ ID NOs: 231-239 |
| V | SEQ ID NOs: 48-52 | SEQ ID NOs: 112-116 | SEQ ID NOs: 176-180 | SEQ ID NOs: 240-244 |
| VI | SEQ ID NOs: 53-64 | SEQ ID NOs: 117-128 | SEQ ID NOs: 181-192 | SEQ ID NOs:245-256 |
| IV | SEQ ID NOs: 257-259 | SEQ ID NOs: 261-263 | SEQ ID NOs: 265-267 | SEQ ID NOs: 269-271 |

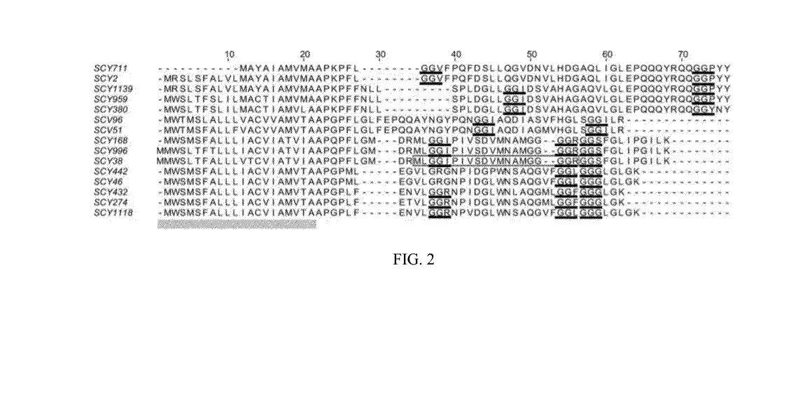

FIG. 2 depicts the alignment of glycine-rich peptides selected from each clade. The signal sequence for processing is represented by the gray bar. The GGx motifs are underlined. The boxed peptide was detected in crude venom proteomic analysis. The following SEQ ID NOs: correspond to the peptide designations contained in the FIG. 1:

TABLE 2
Peptide designations and SEQ ID NOS

| | |
|---|---|
| SCY711 | SEQ ID NO: 73 |
| SCY2 | SEQ ID NO: 101 |
| SCY1139 | SEQ ID NO: 65 |
| SCY959 | SEQ ID NO: 66 |
| SCY380 | SEQ ID NO: 67 |
| SCV96 | SEQ ID NO: 262 |
| SCV51 | SEQ ID NO: 263 |
| SCY168 | SEQ ID NO: 111 |
| SCY996 | SEQ ID NO: 103 |
| SCY38 | SEQ ID NO: 110 |
| SCY442 | SEQ ID NO: 112 |
| SCY46 | SEQ ID NO: 114 |
| SCY432 | SEQ ID NO: 120 |
| SCY274 | SEQ ID NO: 119 |
| SCY1118 | SEQ ID NO: 128 |

A number of conserved amino acid motifs were identified in the peptides associated with the present invention. These conserved motifs include GGG, QQQ, YY, GV, GA, QG, and APXP (SEQ ID NO: 273), wherein X is any amino acid. APXP APXP (SEQ ID NO: 273) is present across all sequences in all clades. Clades I and II contain a common sequence of GA(X)₃GLEPQQQYRQQGGPYY (SEQ ID NO: 274). Clades V and VI contain a common sequence of NPIDG(P/L)WNSAQG(X)₂GGXGGGLG (SEQ ID NOS: 275 and 276). These conserved motifs are consistent with the types of motifs involved with protein folding and elasticity in known silk proteins, such as GGX, GA, QA, GPGXX, GV couplets, QQ, and PXP, and in gluten, YY. In silk proteins, the side chains of di-glutamines (QQ) hydrogen bond to each other forming beta-sheets and beta-turns. The GG's make the folding stronger and more compact. The QQ's are proposed to bond to each other in silk protein formation, as well. The YY's have been shown to cross-link to each other in gluten, making the protein stretchy and elastic. The PXP motif contributes to elasticity in silk.

In certain embodiments, the present invention is directed to a nucleic acid molecule, such as a cDNA, comprising a nucleotide sequence having at a sequence identity of at least 70%, at least 80%, at least 85%, at least 90%, at least 95%, at least 96%, at least 97%, at least 98%, at least 99%, and all integer percentages in between, sequence identity to a cDNA sequence selected from the group consisting of SEQ ID NOs: 1-64 and 129-192. The nucleic acid molecule may also have 100% identity to a cDNA sequence selected from the group consisting of SEQ ID NOs: 1-64 and 129-192. The nucleic acid molecule may comprise a nucleotide sequence having the specified sequence identities to a cDNA sequence selected from any subset of SEQ ID NOs: 1-64 and 129 -192, including SEQ ID NOs: 129-135 (clade I), SEQ ID NOs: 136-166 (clade II), SEQ ID NOs: 167-175 (clade III), SEQ ID NOs: 176-180 (clade V) and SEQ ID NO: 181-192 (clade VI). The nucleic acid molecule of the present invention may specifically exclude SEQ ID NOs: 260 and 268.

In certain embodiments the present invention is directed to a cDNA comprising a nucleotide sequence encoding a peptide having at least 70%, at least 80%, at least 85%, at least 90%, at least 95%, at least 96%, at least 97%, at least 98%, at least 99%, and all integer percentages in between, sequence identity to an amino acid sequence selected from the group consisting of SEQ ID NOs 65-128 and 193-256. The peptide may also have 100% identity to an amino acid selected from the group consisting of SEQ ID NOs: 65-128 and 193-256. The cDNA may comprise a nucleotide sequence encoding a peptide having the specified sequence identities to an amino acid sequence selected from any subset of SEQ ID NOs: 65-128 and 193-256, including SEQ ID NOs: 193-199 (clade I), SEQ ID NOs: 200-230 (clade II), SEQ ID NOs: 231-239 (clade III), SEQ ID NOs: 240-244 (clade V) and SEQ ID NOs: 245-256 (clade VI). The cDNA of the present invention may specifically exclude SEQ ID NOs: 260 and 268.

The present invention is also directed to nucleic acid constructs and recombinant expression vectors that encode the peptides of the present invention. Certain aspects of the invention are directed to nucleic acid constructs and recombinant expression vectors that comprise any of the cDNAs disclosed herein and/or that encode any of the peptides disclosed herein. Certain embodiments of the invention are directed to a nucleic acid construct or recombinant expression vector comprising a nucleotide sequence having at least 70%, at least 80%, at least 85%, at least 90%, at least 95%, at least 96%, at least 97%, at least 98%, at least 99%, and all integer percentages in between, sequence identity to a cDNA sequence selected from the group consisting of SEQ ID NO: 1-64, 129-192, 257-260 and 265-268. The nucleotide sequence may also have 100% sequence identity to a cDNA sequence selected from the group consisting of SEQ ID NO: 1-64, 129-192, 257-260 and 265-268. The nucleic acid construct or recombinant expression vector may comprise a nucleotide sequence having the specified sequence identities to a cDNA sequence selected from any subset of SEQ ID NOs: 1-64, 129-192, 257-260 and 265-268, including SEQ ID NOs: 129-135 (clade I), SEQ ID NOs: 136-166 and 268, (clade II), SEQ ID NOs: 167-175 (clade III), SEQ ID NOs: 176-180 (clade V), SEQ ID NO: 181-192 (clade VI), SEQ. ID. NOs: 265-267 (clade IV).

Certain aspects of the invention are directed to a nucleic acid construct or expression vector comprising a cDNA sequence encoding a peptide having at least 70%, at least 80%, at least 85%, at least 90%, at least 95%, at least 96%, at least 97%, at least 98%, at least 99%, and all integer percentages in between, sequence identity to an amino acid sequence selected from the group consisting of SEQ ID NO: 65-128, 193-256 and 269-272. The nucleic acid construct or recombinant expression vector may comprise a cDNA sequence encoding a peptide having the specified sequence identities to an amino acid sequence selected from any subset of SEQ ID NO: 65-128, 193-256 and 269-272, including SEQ ID NOs: 193-199 (clade I), SEQ ID NOs: 200-230 and 272 (clade II), SEQ ID NOs: 231-239 (clade III), SEQ ID NOs: 240-244 (clade V), SEQ ID NOs: 245-256 (clade VI), and SEQ ID NOs: 269-271 (clade IV).

Several methods exist for expression (e.g. vector and host strain choice) and purification (e.g. osmotic shock, lysis method, fusion protein cleavage, and affinity or non-affinity chromatography resin choices) of the peptides of the present invention. The expression and purification methods of the present invention are designed to allow formation of the final sticky silk or silk-like fiber after purification. Hydrogen bonding and/or aggregation interactions occur co-translationally. Certain embodiments of the invention are directed to the use of a fusion protein for initial expression in the host strain to keep the product soluble and protected in order to avoid killing off the host, or causing the ribosome to stall and signal proteolysis.

Certain embodiments of the present invention are directed to the bacterial expression (in Escherichia coli) of a Scytodes glycine-rich peptide with an N-terminal fusion to a solubilizing protein in order to keep it soluble throughout the purification process. Once the expressed fusion protein is purified, the soluble protein is enzymatically cleaved, and the glycine-rich peptide can be purified away and allowed to fold. The presence of multiple GGx, QQ, and YY motifs within each peptide will drive side chain hydrogen bonding to corresponding motifs in other peptides, forming strong sheets and layering into fiber.

Many expression systems are available and known in the art, including commercially available vectors paired with compatible host strains for optimal expression. Very generally, in certain embodiments, a vector encoding the peptide of interest (and any desired fusion protein components) is created and cloned, the vector is transformed into a host cell, and the host cell is grown in a culture medium under conditions that cause the host cell to express the peptide. The host cells are lysed and the peptides are purified. In embodiments in which a fusion protein is expressed, the other fusion protein components are enzymatically cleaved from the peptide of interest. Exemplary parameters for host cells, growth conditions, cloning and expression vectors, cell lysis and purification are discussed herein.

Host Cell and Growth Conditions

This procedure may be optimized for expression in E.coli host cells, although other host cells can be used. Cell growth and protein expression (see below) can be conducted at temperatures ranging from 18-25° C., or from 18-23° C. Expression using E. coli laboratory strains K12 and DH5α were successful and have been most thoroughly analyzed in connection with the present invention. Other strains, such as BL21, can be used consistent with the present invention. Varying concentrations of IPTG had no effect on cell growth at any of the above temperature ranges. Cultures were grown in LB supplemented with ampicillin (100 μg/ml f.c.)

Cloning Vectors and Expression

Vectors commonly used for cloning and expression are now commercially available and some companies offer services that can cater to custom requests such as the GeneArt division of Invitrogen. A cDNA sequence or peptide sequence of the invention can be used to synthesize the cDNA encoding the peptide of interest using methods known in the art. The cDNA is then subcloned into a suitable expression vector. In one embodiment, the vector has been modified to co-express the peptide of interest and other functional peptides as fusion proteins.

Vectors producing fusion proteins with desired characteristics are known in the art and can be readily identified and obtained by one of ordinary skill in the art. It was determined that use of a fusion peptide comprising a solubilizing protein is desirable in expressing the peptides of the present invention to prevent agglomeration and formation of inclusion bodies.

Solubilizing proteins for use in fusion proteins and vectors producing such fusion proteins are well known in the art and can be readily identified by those in the art. Solubilizing proteins are large soluble proteins that, when coupled to the peptide of interest, maintain the resulting fusion protein soluble and prevent aggregation of the peptide into inclusion bodies. Generally such proteins are over 100 amino acids. Vectors for producing fusion proteins comprising suitable solubilizing proteins are sold by various companies, including Invitrogen and Novagen. Solubilizing proteins include maltose binding protein (MBP), thioredoxin (TRX), glutathione S-transferase (GST), bacterial disulfide oxioreductase (DsbA), bacterial disulfide isomerase (DsbC), N-utilization substance A (NusA), small ubiquitin-like modifier (SUMO), ubiquitin (Ub), bacterioferritin (BFR) and GrpE. Solubilizing proteins MBP, TRX and GST are particularly suitable for use in the fusion protein of the present invention.

The fusion protein may include additional functional peptides. In certain embodiments the fusion protein comprises a periplasm signaling sequence to direct the protein to the periplasm instead of staying in the cytosol. MalE is one periplasm signaling protein suitable for use in the fusion protein of the present invention. Other periplasm signaling proteins are well known in the art.

The fusion protein may also include a purification tag for affinity purification. Six consecutive histidine residues (His₆) is one purification tag suitable for use in the fusion protein of the present invention. Other purification tags are well known in the art.

In the vector, the fusion protein coding sites are preferably separated from the coding site for the peptide of interest by an enzymatic cleavage site to allow for cleavage of the purified peptide of interest from the other components of the fusion protein. A cleavage site for TEV protease is common and suitable for use with the present invention, although other cleavage sites are well known.

Various vectors are suitable for use in expression the peptide and fusion protein of the present invention, including a pET-based vector. Other suitable vectors will be well-known to those in the art. The vector will also include a suitable promoter, such as a T7 inducible promoter affected by IPTG concentrations.

In one embodiment of the invention, the mature cDNA sequence for the mature peptide of interest is synthesized and sub-cloned into a pET-based vector that has been modified to contain a purification tag and two fusion peptides followed by an enzymatic cleavage site for TEV protease (pLicC-MBP) (Cabrita et al., 2006; Klint et al., 2013). Expression is driven by a standard T7 promoter and the lac operator, which can be induced by the presence of IPTG in the medium. This expression system was designed for the expression of cysteine-rich peptides that are expressed in spider venoms, peptides which, if not directed to the periplasm of E. coli during expression, would not fold properly. This construct avoids co-translational aggregates (e.g., inclusion bodies) that are virtually impossible to tease apart once formed. The fusion construct is as follows:

-(MalEss)-His6-MBP-(TEVsite)-SCY38-

The MalE signal sequence (MalEss) targets the fusion protein to the periplasm of E. coli; the six consecutive histidine residues (His6) are for affinity purification; the maltose binding protein (MBP) is a solubilizing protein big enough to keep the peptide from improperly folding; the TEV recognition site is for cleaving the purified peptide from the fusion protein. This design defers intermolecular hydrogen bonding until later in the purification process.

Induction with IPTG is most successful using low concentrations (<10 μM), although higher concentrations of IPTG (50-1000 μM), or higher, can be used. Induction times between 4-24 h in the presence of IPTG at 18-23° C. are consistent with the present invention.

Use of one vector lacking a periplasmic targeting protein and soluble protein, resulted in unsuccessful purification. A fusion construct of -His6-(TEVsite)-SCY38 in a pET-based vector aggregated as inclusion bodies in any bacterial strain used, and no amount of washing or dilution of buffer could tease the aggregated product apart. This same result was observed under IPTG concentrations ranging from 10-1000 μM, spanning a range of growth temperatures (16-25° C.), and even in the presence of DMSO (4% and 40% v/v) added to a cell pellet from 10 μM IPTG induction.

Cell Lysis and Purification

In one exemplary embodiment, induced cells are grown through late log phase (OD₆₀₀ 0.8-1.2), then pelleted by centrifugation, according to standard procedures. Centrifugation for 15 min at 3400 rpm at 10° C. is consistent with the present invention. Pellets should be stored at −20° C. On the day of purification, each pellet is thawed on ice and resuspended in 2.5 ml of BugBuster protein extraction reagent (Novagen) and 1 μM of benzoase nuclease (Novagen). Protease inhibitors are not necessary. Tubes are incubated, rocking slowly for 20 mM. Lysates are centrifuged at 2,000×g for 30 min. Supernatants (soluble lysates) are then loaded on to a Ni-NTA His-Bind column equilibrated according to the manufacturer's protocol (Novagen). Purification may be performed at room temperature by gravity flow. It will be readily understood by one in the art that reagents, conditions, amounts and times may be modified consistent with the present invention.

The eluted fusion protein can either be directly cleaved with TEV protease or concentrated first, such as by using an Amicon filter (EMD Millipore), and then cleaved.

Successful cleavage of the purified fusion protein is influenced by buffer constitution, reaction temperature and reaction time. First, imidazole would be either removed or diluted from the buffer that the purified fusion protein is in after elution from the column; this can be done either with dialysis, column chromatography, or filter centrifugation (e.g. Amicon or Centricon, EMD Millipore). Using one of these methods will also de-salt and remove small contaminants from the buffer, further removing any molecules that may interfere with the cleavage reaction.

Instead of using the suggested elution buffer (1×) for the final step of purification, a diluted form would be used so the imidazole concentration is ≦100 mM f.c; for this, the elution buffer (1×) can be mixed with the binding buffer (1×) in a 1:10 ratio (v/v). The Amicon Ultra filter or dialysis systems (EMD Millipore) are fast and convenient for de-salting the elution buffer, which also contains NaCl.

The peptide of interest is then cleaved from the fusion protein using an enzyme designed to cleave at the included cleavage site. TEV (tobacco etch virus) protease is an enzyme commercially available and commonly used to cleave a specific amino acid motif (Glu-Asn-Leu-Tyr-Phe-Gln-Gly, cleaving between Gln and Gly); it was designed in the vector described above to link the soluble His-MBP to the peptide of interest. Although the optimal temperature for cleavage is 30° C., the AcTEV protease (Invitrogen) works with high efficiency across a range of temperatures and in a pH range from 6-8.5. Suitable conditions for other cleavage enzymes are known in the art.

The cleavage reaction should be rocking slowly at room temperature. As the peptides are separated from the soluble fusion protein, hydrogen bonding will attract peptides to each other and drive the formation of a fiber. The peptides will be the adhesive and contractile strands of the fibers. Based on the positions of the motifs (GGX), peptides will align to form β-sheets and/or turns that stack tightly and repeatedly into a strong fiber. In addition, the side chains of QQ and YY motifs will also find each other to form extensive webs of hydrogen bonding networks. Water content may need to be adjusted with respect to super-contraction, as the hydrogen bonds may re-arrange in response to an increase in humidity. Finally, proline residues will contribute to elasticity, depending on their ration with the glycines present in the peptides.

Certain embodiments of the invention are directed to products produced using the peptides of the invention. The peptides of the invention and fibers produced from the peptides of the invention can be incorporated into articles to impart strength to the articles. Articles that may be formed using the peptides and fibers described herein include armor and car doors, fabric, clothing, internal stitches, capsules for drug delivery, nerve repair products, and ligament replacement products.

Certain exemplary embodiments are illustrated by the following non-limiting example.

### EXAMPLE 1

SCY38 (GenBank ID KF860355) SEQ ID NO: 46 was chosen for expression and purification because it was detected in both transcriptome and the proteome analyses. Detection in both systems means that the SCY38 mRNA sequence was identified in the venom gland tissue and its corresponding translated peptide was identified in the crude venom milked from the same spiders.

The full-length cDNA sequence for SCY38 is listed as SEQ ID NO: 46. The corresponding full length peptide sequence is listed SEQ ID NO: 110. The full-length peptide sequence is predicted to contain an N-terminal signal sequence for processing, and the resulting mature peptide sequence is listed as SEQ ID NO: 238, as:

(SEQ ID NO: 238)
APQPFLGMDRMLGGIPIVSDVMNAMGGGGRGGSFGLIPGILK.

Glycine residues constitute 26% of the mature peptide (above in bold).

Recombinant Expression of SCY38

pLiCC-SCY38 Fusion Protein

The mature cDNA coding sequence for the mature peptide sequence of SCY38 was synthesized and sub-cloned into the pLiCC expression vector by GeneArt (Invitrogen). This is a pET-based vector that has been modified to contain two fusion proteins followed by an enzymatic cleavage site for TEV protease (pLicC-MBP) (Cabrita et al., 2006; Klint et al., 2013). Expression is driven by a standard T7 promoter and the lac operator, which can be induced by the presence of IPTG in the medium. This expression system was designed for the expression of cysteine-rich peptides that are expressed in spider venoms, peptides which, if not directed to the periplasm of E. coli during expression, would not fold properly. This fusion construct was chosen for the expression of glycine-rich peptides in order to avoid co-translational aggregates (e.g., inclusion bodies) that are virtually impossible to tease apart once formed. The fusion construct is as follows:

-(MalEss)-His₆-MBP-(TEVsite)-SCY38-

The MalE signal sequence (MalEss) targets the fusion protein to the periplasm of E. coli; the six consecutive histidine residues (His6) are for affinity purification; the maltose binding protein (MBP) is a solubilizing protein big enough to keep the peptide from improperly folding; the TEV recognition site is for cleaving the purified peptide from the fusion protein. This design defers intermolecular hydrogen bonding until later in the purification process.

Cell Growth and Induction of Expression

To grow cell transformants, a single colony was inoculated into 2 ml LB containing ampicillin (100 μg/ml f.c.) (LBamp) and grown overnight at 22° C. shaking at 225 rpm. The following morning, 1 ml of the overnight culture was inoculated into 100 ml of LBamp and grown to early log phase (OD₆₀₀ 0.4) at 22° C. shaking at 225 rpm. Recombinant expression of the fusion protein was induced by adding IPTG (10 μM f.c.), and cells were grown for 24 h at 22° C. shaking at 225 rpm. Cultures were divided into 50 ml centrifuge tubes and centrifuged at 3400 rpm for 20 min at 10° C. The supernatant was discarded and the cell pellets were placed at −20° C. overnight or until ready for purification. The average weight of the cell pastes was 0.47 g.

Cell Lysis and Purification of the Fusion Protein

A single pellet (from −20° C.) was thawed on ice and resuspended in 2.5 ml of BugBuster (Novagen, EMD Millipore). The cell suspension was divided between two 1.5 ml μfuge tubes and 1 μl of Benzoase Nuclease (Novagen, EMD Millipore) was added to each. This can be scaled up accordingly.

The mixtures (lysates) were incubated, rocking slowly on a rotating platform for 15 min. The tubes were centrifuged for 20 min at 2,000×g. The supernatant is the “soluble lysate”, and was transferred to a fresh tube containing resin (His-Bind Purification kit, EMD Millipore) that was charged according to the manufacturer's protocol. The resin-lysate mixture was inverted to mix, and then incubated for 5 min. Following the manufacturer's protocol and kit buffers, the bound resin was washed and the fusion protein was eluted (eluate). The “inclusion body” (IB) protocol was followed, which involved a series of washes with 1:10 BugBuster; the supernatant from each centrifuge step was removed and saved, and the pellet from that spin was washed repeatedly up to five times. The supernatants were analyzed as IB1-4 and fifth (IB5) was the complete mix.

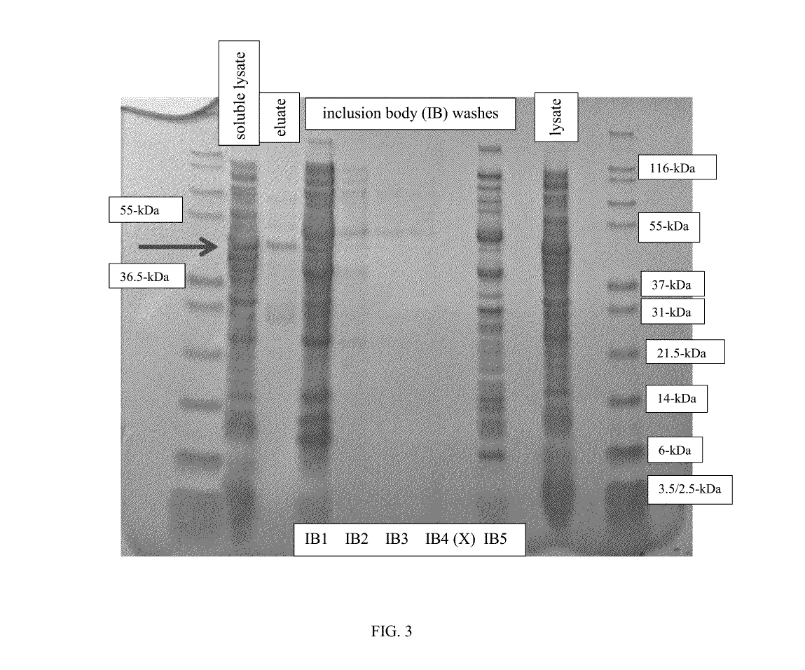

After each purification step, 10 μl was removed, mixed with 1 vol of 2× SDS-PAGE sample buffer with reducing agent (Invitrogen). All samples were placed at 85° C. for 5 min, placed on ice, and centrifuged briefly (˜5 s). Samples were analyzed using SDS-PAGE (10% tris-glycine). Gels were stained using Coomassie SimpleBlue (Invitrogen) and destained using H₂O. The fusion protein is predicted to be ˜50-kDa, which was detected in the lysate, soluble lysate, and eluate fractions; it was not detected in any of the IB washes, as shown in FIG. 3.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

### REFERENCES

Boutry, C. and Blackledge, T. A. (2010) Evolution of supercontraction in spider silk: structure-function relationship from tarantulas to orb-weavers. J. Exp. Biol. 213:3505-3514.

Cabrita, L. D., Dai, W., and Bottomley, S. P. (2006) A family of E. coli expression vectors for laboratory scale and high throughput soluble protein production. BMC Biotechnology 6:12 DOI: 10.1186/1472-6750/6/12.

Clements, R. and Li, D. Q. (2005) Regulation and non-toxicity of the spit from the pale spitting spider Scytodes pallida (Araneae: Scytodidae). Ethology 111:311-321.

Foelix, R. F. Biology of Spiders. 2nd edition ed.; Oxford University Press: New York, 1996.

Gosline, J. M., Guerette, P. A., Ortlepp, C. S., and Savage, K. N. (1999) The mechanical design of spider silks: from fibroin sequence to mechanical function. J. Exp. Biol. 202:3295-3303.

Gosline, J. M., Lillie, M., Carrington, E., Guerette, P. A., Ortlepp, C. S., and Savage, K. N. (2002) Elastic proteins: biological roles and mechanical properties. Phil. Trans. R. Soc. Lond. B 357:121-132.

Klint, J. K., Senff, S., Saez, N. J., Seshadri, R., Lau, H. Y., Bende, N. S., Undheim, E. A., B., Rash, L. D., Mobli, M., and King, G. F. (2013) Production of recombinant disulfide-rich venom peptides for structural and functional analysis via expression in the periplasm of E. coli. PLoS ONE 8:e63865 DOI: 10.1371/journal.pone.0063865.

Kluge, J. A., Rabotyagova, O., Leisk, G. G., and Kaplan, D. L. (2008) Spider silks and their applications Trends in Biotechnology 26:244-251.

Perry, D. J., Bittencourt, D., Siltberg-Liberles, J., Rech, E. L., Lewis, R. V. (2010) Piriform spider silk sequences reveal unique repetitive elements. Biomacromolecules 11:3000-3006.

R\ddot{8}mer, L. and Scheibel, T. (2008) The elaborate structure of spider silk: structure and function of a natural high performance fiber. Prion 2:154-161.

Sahni, V., Blackledge, T. A., and Dhinojwala, A. (2011) Changes in the adhesive properties of spider aggregate glue during the evolution of cobwebs. Scientific Rep. 1:41 DOI:10.1038/srep00041.

Savage, K. N. and Gosline, J. M. (2008) The role of proline in the elastic mechanism of hydrated spider silks. J Exp. Biol. 211:1948-1957.

Stark, M., Grip, S., Rising, A., Hedhammar, M., Engstrom, W., Hjälm, G., and Johansson, J. (2007) Macroscopic fibers self-assembled from recombinant miniature spider silk proteins. Biomacromolecules 8:1695-1701.

Suter, R. B. and Stratton, G. E. (2005) Scytodes vs. Schizocosa: predatory techniques and their morphological correlates. J. Arachnol. 33:7-15.

Suter, R. B. and Stratton, G. E. (2009) Spitting performance parameters and their biomechanical implications in the spitting spider, Scytodes thoracica. J. Insect Sci. 9:1-15.

Swanson, B. O., Blackledge, T. A., Summers, A. P., and Hayashi, C. Y. (2006) Spider dragline silk: correlated and mosaic evolution in high-performance biological materials. Evolution 60:2539-2551.

Vasanthavada, K., Hu, X., Tuton-Blasingame, T., Hsia, Y., Sampath, S., Pacheco, R., Freeark, J., Falick, A. M., Tang, S., Fong, J., Kohler, K., La Mattina-Hawkins, C., Vierra, C. (2012) Spider glue proteins have distinct architectures compared with traditional spidroin family members. J Biol. Chem. 287:35986-35999.

World Spider Catalog (2015). World Spider Catalog. Natural History Museum Bern, online at http://wsc.nmbe.ch, version 15.5, accessed on Jan. 7, 2015.

Zobel-Thropp, P. A., Correa, S. M., Garb, J. E. and Binford, G. J. (2014) Spit and venom from Scytodes spiders: a diverse and distinct cocktail J. Proteome Res. 13: 817-835 DOI: 10.1021/pr400875s.

What is claimed and desired to be secured by Letters Patent is as follows:

1. A cDNA comprising a nucleotide sequence having at least 95% sequence identity to a cDNA sequence selected from the group consisting of SEQ ID NOS: 1-64 and 129-192.

2. The cDNA of claim 1, wherein the cDNA sequence is selected from the group consisting of SEQ ID NOS: 129-135.

3. The cDNA of claim 1, wherein the cDNA sequence is selected from the group consisting of SEQ ID NOS: 136-166.

4. The cDNA of claim 1, wherein the cDNA sequence is selected from the group consisting of SEQ ID NOS: 167-175.

5. The cDNA of claim 1, wherein the cDNA sequence is selected from the group consisting of SEQ ID NOS: 176-180.

6. The cDNA of claim 1, wherein the cDNA sequence is selected from the group consisting of SEQ ID NOS: 181-192.

7. The cDNA of claim 1, wherein the cDNA sequence has at least 97% sequence identity to the cDNA sequence selected from the group consisting of SEQ ID NOS: 129-192.

8. A recombinant expression vector comprising the cDNA of claim 1.

9. A vector comprising a nucleotide sequence having at least 95% sequence identity to a cDNA sequence selected from the group consisting of SEQ ID NOS: 1-64, 129-192, 257-260 and 265-268.

10. The vector of claim 9, wherein the cDNA sequence is selected from the group consisting of SEQ ID NOS: 129-135.

11. The vector of claim 9, wherein the cDNA sequence is selected from the group consisting of SEQ ID NOS: 136-166 and 268.

12. The vector of claim 9, wherein the cDNA sequence is selected from the group consisting of SEQ ID NOS: 167-175.

13. The vector of claim 9, wherein the cDNA sequence is selected from the group consisting of SEQ ID NOS: 176-180.

14. The vector of claim 9, wherein the cDNA sequence is selected from the group consisting of SEQ ID NOS: 181-192.

15. The vector of claim 9, wherein the cDNA sequence is selected from the group consisting of SEQ ID NOS: 265-267

16. A cDNA comprising a nucleotide sequence encoding a peptide having at least 95% sequence identity to an amino acid sequence selected from the group consisting of SEQ ID NOS: 65-128 and 193-256.

17. A vector comprising the cDNA of claim 16.

18. A vector comprising a cDNA sequence encoding a peptide having at least 95% sequence identity to an amino acid sequence selected from the group consisting of SEQ ID NOS: 65-128, 193-256, 261-264, and 269-272.

19. The vector of claim 18, wherein the amino acid sequence is selected from the group consisting of SEQ ID NOS: 193-199.

20. The vector of claim 18, wherein the amino acid sequence is selected from the group consisting of SEQ ID NOS: 200-230 and 272.

21. The vector of claim 18, wherein the amino acid sequence is selected from the group consisting of SEQ ID NOS: 231-239.

22. The vector of claim 18, wherein the amino acid sequence is selected from the group consisting of SEQ ID NOS: 240-244.

23. The vector of claim 18, wherein the amino acid sequence is selected from the group consisting of SEQ ID NOS: 245-256.

24. The vector of claim 18, wherein the amino acid sequence is selected from the group consisting of SEQ ID NOS: 269-271.

25. The vector of claim 18, wherein the peptide encoded by the cDNA sequence has at least 97% sequence identity to the amino acid sequence selected from the group consisting of SEQ ID NOS: 193-256 and 269-272.

26. The vector of claim 18, wherein the peptide encoded by the cDNA sequence comprises the amino acid sequence selected from the group consisting of SEQ ID NOS: 193-256 and 269-272.

27. A host cell comprising the vector of claim 18.

28. The vector of claim 18, further comprising a sequence encoding a solubilizing protein.

29. A host cell comprising the nucleic acid construct or expression vector of claim 28.

30. A fusion peptide comprising a solubilizing peptide and a peptide having at least 95% sequence identity to an amino acid sequence selected from the group consisting of SEQ ID NOS: 193-256 and 269-272.

31. The fusion peptide of claim 30, wherein the solubilizing peptide is selected from the group consisting of maltose binding protein, thioredoxin, glutathione S-transferase.

32. The fusion peptide of claim 30, further comprising a purification tag.

33. The fusion peptide of claim 30, further comprising a periplasm signaling peptide.

34. A vector comprising a cDNA sequence encoding a peptide having at least 85% sequence identity to an amino acid sequence selected from the group consisting of SEQ ID NO: 65-71, 72-102, 103-111, 112-116, 117-128, 261-263, 264, 193-199, 200-230, 231-239, 240-244, 245-256, 269-271 and 272 and having at least two sequence motifs selected from the group consisting of alanine-proline-X-proline (SEQ ID NO: 273), glycine-glycine-glycine, glutamine-glutamine-glutamine, tyrosine-tyrosine, glycine-valine, glycine-alanine and glutamine-glycine, wherein X is any amino acid, and said peptide is a spider venom fiber peptide.

35. The vector of claim 34, wherein one of said sequence motifs is alanine-proline-X-proline (SEQ ID NO: 273).

36. A method for production of a spider venom fiber peptide comprising:
growing the host cell of claim 27 in a culture medium under conditions that cause the host cell to express the peptide.

37. A method for production of a spider venom fiber peptide:
growing the host cell of claim 29 in a culture medium under conditions that cause the host cell to express the peptide; and
enzymatically cleaving the solubilizing protein from the peptide.


SEQUENCE LISTING (276 sequences)

SEQ ID NO: 1 - DNA, 201 bp, Artificial Sequence
  Synthetic
atgtggtctcttactttttccctgattttgatggcttgtactattgccatggtcttggcagctcccaaaccgtttttcaacttactcagtccgcttgatggtttattaggaggaatagatagtgtggcgcatgcaggagcacaagttcttggccttgaaccacagcagcagtacaggcaacaagggggctacaactactga

SEQ ID NO: 2 - DNA, 201 bp, Artificial Sequence
  Synthetic
atgtggtctcttactttttccctgattttgatggcttgtactattgccatggtcatggcagctcccaaaccgtttttcaacttactcagtccgcttgatggtttattaggaggaatagatagtgtggcgcatgcaggagcacaagttcttggccttgaaccacagcagcagtacaggcaacgagggggctacaactactga

SEQ ID NO: 3 - DNA, 201 bp, Artificial Sequence
  Synthetic
atgtggtctcttactttttccctgattttgatggcttgtactattgccatggtcatggcagctcccaaaccgtttttcaacttactcagtccgtttgatggtttattaggaggaatagatagtgtggcgcatgcaggagcacaagttcttggccttgaaccacagcagcagtacaggcaacaagggggctacaactactga

SEQ ID NO: 4 - DNA, 201 bp, Artificial Sequence
  Synthetic
atgtggtctcttactttttccctgattttgatggcttgtactattgccatggtcatggcagctcccaaaccgttttccaacttactcagtccgcttgatggtttattaggaggaatagatagtgtggcgcatgcaggagcacaagttcttggccttgaaccacagcagcagtacaggcaacaagggggctacaactactga

SEQ ID NO: 5 - DNA, 201 bp, Artificial Sequence
  Synthetic
atgtggtctcttactttttccctgattttgatggcttgtactattgccatggtcatggcagctcccaaaccgtttttcaacttactcagtccgcttgatggtttattaggaggaatagatagtgtggcgcatgcaggagcacaagttcttggccttgaaccacagcagcagtacaggcaacaagggggctacaactactga

SEQ ID NO: 6 - DNA, 201 bp, Artificial Sequence
  Synthetic
atgcggtctctgtcttttgccctagttttgatggcttatgctattgccatggtcatggcagctcccaaaccgtttttcaacttactcagtccgcttgatggtttattaggaggaatagatagtgtggcgcatgcaggagcacaagttcttggccttgaaccacagcagcagtacaggcaacaagggggcccttactactaa

SEQ ID NO: 7 - DNA, 201 bp, Artificial Sequence
  Synthetic
atgtggtctcttactttttccctgattttgatggcttgtactattgccatggtcatggcagctcccaaaccgtttttcaacttactcagtccgcttgatggtttattaggaggaatagatagtgtggcgcatgcaggagcacaagttcttggccttgaaccacagcagcagtacaggcaacaagggggcccttactactaa

SEQ ID NO: 8 - DNA, 168 bp, Artificial Sequence
  Synthetic
atgctattgccatggtcatggcagctcccaaaccatttttgggggggagtttttccccagtttgatagtttattacaaggagtagataatgtgttgcatgatggagcacaacttattggccttgaaccacagcagcagtacaggcaacaagggggcccttactactaa

SEQ ID NO: 9 - DNA, 174 bp, Artificial Sequence
  Synthetic
atggcttatgctattgccatggtcatggcagctcccaaaccatttttggggggagtttttccgcagtttgatagtttattacaaggagtagataatgtgttgcatgatggagcacaacttattggccttgaaccacagcagcagtacaggcaacaagggggcccttactactaa

SEQ ID NO: 10 - DNA, 174 bp, Artificial Sequence
  Synthetic
atggcttatgctattgccatggtcatggcagctcccaaaccatttttggggggagtctttccgcagtttgatagtttattacaaggagtagatgatgtgttgcatgatggagcacaacttattggccttgaaccacagcagcagtacaggcaacaagggggcccttactactaa

SEQ ID NO: 11 - DNA, 204 bp, Artificial Sequence
  Synthetic
atgcggtctctgtcttttgccctagcttttgatggctatgctattgccatggtcatggcagctcccaaaccatttttggggggagtttttccgcagtttgatagtttattacaaggagtagataatgtgttgcatgatggagcacgacttattggccttgaaccacagcagcagtacaggcaacaagggggcccttactactaa

SEQ ID NO: 12 - DNA, 204 bp, Artificial Sequence
  Synthetic
atgcggtctctgtcttttgccctagttttgatggcttatgctattgccatggtcatggcagctcccaaaccatttttgggggcagtttttccgcagtttgatagtttattacgaggagtagataatgtgttgcatgatggagcacaacttattggccttgaaccacagcagcagtacaggcaacaagggggcccttactactaa

SEQ ID NO: 13 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagttcattacaaggagtagataatgtgttgcatgatggagcacaacttattggccttgaaccacagcagcagtacaggcaacaagggggcccttactactaa

SEQ ID NO: 14 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataacgtgttgcatgatggagcacaacttattggccttgaaccacagcagccgtacaggcaacaagggggcccttactactaa

SEQ ID NO: 15 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgatggagcacaacttattggccttgaaccacagcagcagtacaggcaacaagggggcccttgctactaa

SEQ ID NO: 16 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgagttgcatgatggagcacaacttattggccttgaaccacagcagcagtacaggcaacaagggggcccttactactaa

SEQ ID NO: 17 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgatggagtacaacttattggccttgaaccacagcagcagtacaggcaacaagggggcccttactactaa

SEQ ID NO: 18 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaagtagtagataatgtgttgcatgatggagcacaacttattggccttgaaccacagcagcagtacaggcaacaagggggcccttactactaa

SEQ ID NO: 19 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgatggagcacaatttattggccttgaaccacagcagcagtacaggcaacaagggggcccttactactaa

SEQ ID NO: 20 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgatggagcacaactttttggccttgaaccacagcagcagtacaggcaacaagggggcccttactactaa

SEQ ID NO: 21 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgatggagcacaacttattggccttgaaccacagcggcagtacaggcaacaagggggcccttactactaa

SEQ ID NO: 22 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgatggagcacaacttattggccttgaaccacagcagcagtgcaggcaacaagggggcccttactactaa

SEQ ID NO: 23 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataaagtgttgcatgatggagcacaacttattggccttgaaccacagcagcagtacaggcaacaagggggcccttactactaa

SEQ ID NO: 24 - DNA, 204 bp, Artificial Sequence
  Synthetic
atgcggtctctgtcttttgccctagttttgatggcttatgctattgccatggtcgtggcagctcccaaaccatttttggggggagtttttccgcagtttgatagtttattacaaggagtagataatgtgttgcgtgatggagcacaacttattggccttgaaccacagcagcagtacaggcaacaagggggcccttactactaa

SEQ ID NO: 25 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcgtgatggagcacaacttattggccttgaaccacagcagcagtacaggcaacaagggggcccttactactaa

SEQ ID NO: 26 - DNA, 204 bp, Artificial Sequence
  Synthetic
atgcggtctctgtcttttgccctagttttgttggcttatgctattgccatggtcatggcagctcccaaaccatttttggggggagtttttccgcagtttgatagtttattacaaggagtagataatgtgttgcatgatggagcacaacttattggccttgaaccacagcagcagtacaggcaacaagggggcccttactactaa

SEQ ID NO: 27 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtaaataatgtgttgcatgatggagcacaacttattggccttgaaccacagcagcagtacaggcaacaagggggcccttactactaa

SEQ ID NO: 28 - DNA, 204 bp, Artificial Sequence
  Synthetic
atgcggtccctgtcttttgccctagttttgatggcttatgctattgccatggtcatggcagctcccaaaccatttttggggggagtttttccgcagtttgatagtttattacaaggagtagataatgtgttgcatgatggagcacaacttattggccttgaaccacagcagcagtacaggcaacaagggggcccttaccactaa

SEQ ID NO: 29 - DNA, 204 bp, Artificial Sequence
  Synthetic
atgcggtctctgtcttttgccctatttttgatggcttatgctattgccatggtcatggcagctcccaaaccatttttggggggagtttttccgcagtttgatagtttattacaaggagtagataatgtgttgcatgatggagcacaacttattggccttgaaccacagcagcagtacaggcaacaagggggcccttactactaa

SEQ ID NO: 30 - DNA, 204 bp, Artificial Sequence
  Synthetic
atgcggtctctgtcttttgccctagttttgacggcttatgctattgccatggtcatggcagctcccaaaccatttttggggggagtgtttccgcagtttgatagtttattacaaggagtagataatgtgttgcatgatggagcacaacttattggccttgaaccacagcagcagtacaggcaacaagggggcccttactactaa

SEQ ID NO: 31 - DNA, 204 bp, Artificial Sequence
  Synthetic
atgcggtctctgtcttttgccctagttttgatggcttatgctattgccatggtcatggcagctcccaaaccatttttggggggagtttttccgctgtttgatagtttattacaaggagtagataatgtgttgcatgatggagcacaacttattggccttgaaccacagcagcagtacaggcaacaagggggcccttactactaa

SEQ ID NO: 32 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgcaggagcacaacttattggccttgaaccacagcagcagtacaggcaacaagggggcccttactactaa

SEQ ID NO: 33 - DNA, 204 bp, Artificial Sequence
  Synthetic
atgcggtctctgtcttttgccctagttttgatggcttatgctattgccatggtcacggcagctcccaaaccatttttggggggagtttttccgcagtttgatagtttattacaaggagtagataatgtgttgcatgatggagcacaacttattggccttgaaccacagcagcagtacaggcaacaagggggcccttactactaa

SEQ ID NO: 34 - DNA, 204 bp, Artificial Sequence
  Synthetic
atgcggtctctgtcttttgccctagttttgatggcttatgctattgccatggccatggcagctcccaaaccatttttggggggagtttttccgcagtttgatagtttattacaaggagtagataatgtgttgcatgatggagcacaacttattggccttgaaccacagcagcagtacaggcaacaagggggcccttactactaa

SEQ ID NO: 35 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagatgatgtgttgcatgatggagcacaacttattggccttgaaccacagcagcagtacaggcaacaagggggcccttactactaa

SEQ ID NO: 36 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgatggagcacaacttgttggccttgaaccacagcagcagtacaggcaacaagggggcccttactactaa

SEQ ID NO: 37 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtagataatgtgttgcatgatggagcacaacttattggccttgaaccacagcagcagtacaggcaacaagggggcccttactactaa

SEQ ID NO: 38 - DNA, 204 bp, Artificial Sequence
  Synthetic
atgtggtctcttactttttccctgattttgatggcttgtactattgccatggtcatggcagctcccaaaccatttttggggggagtttttccgcagtttgatagtttattacaaggagtagataatgtgttgcatgatggagcacaacttattggccttgaaccacagcagcagtacaggcaacaagggggcccttactactaa

SEQ ID NO: 39 - DNA, 192 bp, Artificial Sequence
  Synthetic
atgatgtggtctctgacattcaccctgcttttgatcgcttgtgttattgccacggttatagctgctccccaaccctttcttggaatggatagaatgctaggtggtataccaattgtcagtgatgtgatgaatgcaatgggcggcggcggccgcggcggtagcttcgggctcatccccgggatcctaaaatag

SEQ ID NO: 40 - DNA, 192 bp, Artificial Sequence
  Synthetic
atgatgtggtctctgacgtttgccctacttttggtcacttgtgttattgccacggttatagctgctccccaaccctttcttggaatggatagagtgctaggtggtataccaattgtcagtgatgtgatgaatgcaatgggcggcggcggccgcggcggtagcttcgggctcatccccgggatcctaaaatag

SEQ ID NO: 41 - DNA, 192 bp, Artificial Sequence
  Synthetic
atgacgtggtctctgacgtttgccctacttttggtcacttgtgttattgccacggttatagctgctccccaaccctttcttggaatggatagaatgctaggtggtataccaattgtcagtgatgtgatgaatgcaatgggcggcggcggccgcggcggtagcttcgggctcatccccgggatcctaaaatag

SEQ ID NO: 42 - DNA, 192 bp, Artificial Sequence
  Synthetic
atgatgtggtctctgacgtttgccctacttttggtcactcgtgttattgccacggttatagctgctccccaaccctttcttggaatggatagaatgctaggtggtataccaattgtcagtgatgtgatgaatgcaatgggcggcggcggccgcggcggtagcttcgggctcatccccgggatcctaaaatag

SEQ ID NO: 43 - DNA, 192 bp, Artificial Sequence
  Synthetic
atgatgtggtctctgacgtttgccctacttttggtcacttgtgttattgccacggttatagctgctccccaaccctttcttggaatggatagaatgctaggtggtataccaattgtcagtgatgcgatgaatgcaatgggcggcggcggccgcggcggtagcttcgggctcatccccgggatcctaaaatag

SEQ ID NO: 44 - DNA, 192 bp, Artificial Sequence
  Synthetic
atgatgtggtctctgacgtttgccctacttttggtcacttgtgttattgccacggttatagctgctccccaaccctctcttggaatggatagaatgctaggtggtataccaatcgtcagtgatgtgatgaatgcaatgggcggcggcggccgcggcggtagcttcgggctcatccccgggatcctaaaatag

SEQ ID NO: 45 - DNA, 192 bp, Artificial Sequence
  Synthetic
atgatgtggtctctgacgtttgccctacttttggtcacttgtgttattgccacggttatagctgctccccaaccctttcttggaatggatagaatgctaggtggtataccaattgtcagtgatgtgatgaatgcaatgggcggcggcggccgcggccgtagcttcgggctcatccccgggatcctaaaatag

SEQ ID NO: 46 - DNA, 192 bp, Artificial Sequence
  Synthetic
atgatgtggtctctgacgtttgccctacttttggtcacttgtgttattgccacggttatagctgctccccaaccctttcttggaatggatagaatgctaggtggtataccaattgtcagtgatgtgatgaatgcaatgggcggcggcggccgcggcggtagcttcgggctcatccccgggatcctaaaatag

SEQ ID NO: 47 - DNA, 189 bp, Artificial Sequence
  Synthetic
atgtggtcaatgtcttttgccttgcttttgatcgcttgtgtcattgccacggttatagctgctccccaaccctttcttggaatggatagaatgctaggtggtataccaattgtcagtgatgtgatgaatgcaatgggcggcggcggccgcggcggtagcttcgggctcatccccgggatcctaaaatag

SEQ ID NO: 48 - DNA, 177 bp, Artificial Sequence
  Synthetic
atgtggtcaatgtcttttgccttgcttttgatcgcttgtgtcattgccatggtcacggctgctcctggacctatgcttgagggtgtattgggtcgtggaaatccgatcgatggaccatggaattcagcacaaggtgtgtttggtggtttgggcggtggcctcggccttggaaaatga

SEQ ID NO: 49 - DNA, 177 bp, Artificial Sequence
  Synthetic
atgtggtcaatgtcttttgcctcgcttttgatcgcttgtgtcattgccatggtcacggctgctcctggacctatgcttgagggtgtattgggtcgtggaaatccgatcgatggactatggaattcagcacaaggtgtgtttggtggtttgggcggtggcctcggccttggaaaatga

SEQ ID NO: 50 - DNA, 177 bp, Artificial Sequence
  Synthetic
atgtggtcaatgtcttttgccttgcttttgatcgcttgtgtcattgccatggtcacggctgctcctggacctatgcttgagggtgtattgggtcgtggaaatccgatcgatggactatggaattcagcacaaggtgtgtttggtggtttgggcggtggcctcggccttggaaaatga

SEQ ID NO: 51 - DNA, 177 bp, Artificial Sequence; identical to SEQ ID NO: 50
  Synthetic

SEQ ID NO: 52 - DNA, 177 bp, Artificial Sequence
  Synthetic
atgtggtcaatgtcttttgccttgcttttgatcgcttgtgtcattgccatggtcacggctgctcctggacctatgcttgagggtgtattgggtcgtggaaatccgatcgatggactatggaattcagcacaaggtgtgtttggtggtttgggcggtggcctcggccttgtaaaatga

SEQ ID NO: 53 - DNA, 177 bp, Artificial Sequence
  Synthetic
atgtggtcaatgtcttttgccttgcttctgatcgcttgtgtcattgccatggtcacggctgctcctggaccattatttgagaatgtattgggtggtagaaatccggtcgatggactatggaattcagcacaaggtgtgtttggtggtttgggcggtggcctcggccttggaaaatga

SEQ ID NO: 54 - DNA, 162 bp, Artificial Sequence
  Synthetic
atgtcttttgccttgcttttgatcgcttgtgtcattgccatggtcacggctgctcctggaccattatttgagaaagtattgggtggtagaaatccgatcgatggactatggaattcagcacaaggtatgcttggtggctttggcggtggccttggaaaatga

SEQ ID NO: 55 - DNA, 171 bp, Artificial Sequence
  Synthetic
atgtggtcaatgtcttttgccttgcttttgatcgcttgtgtcattgccatggccacggctgctcctggaccattatttgagaatgtattgggtggtagaaatccgatcgatggactatggaattcagcacaaggtatgcttggtggctttggcggtggccttggaaaatga

SEQ ID NO: 56 - DNA, 171 bp, Artificial Sequence
  Synthetic
atgtggtcaatgtcttttgccttgcttttgatcgcttgtgtcattgccatggtcacggctgctcctggaccattatttgagactgtattgggtggtagaaatccgatcgatggactatggaattcagcacaaggtatgcttggtggctttggcggtggccttggaaaatga

SEQ ID NO: 57 - DNA, 171 bp, Artificial Sequence
  Synthetic
atgtggtcaatgtcttttgccttgcttttgatcgcttgtgtcattgccatggtcacggctgctcctggaccattatttgagaatgtattgggtggtagaaatccgatcgatggactatggaatttagcacaaggtatgcttggtggctttggcggtggccttggaaaatga

SEQ ID NO: 58 - DNA, 171 bp, Artificial Sequence
  Synthetic
atgtggtcaatgtcttttgccttgcttttgatcgcttgtgtcattgccatggccacggctgctcctggaccattatttgagaatgcattgggtggtagaaatccgatcgatggactatggaattcagcacaaggtatgcttggtggctttggcggtggccttggaagatga

SEQ ID NO: 59 - DNA, 171 bp, Artificial Sequence
  Synthetic
atggggtcaatgtcttttgccttgcttttgatcgcttgtgtcattgccatggtcacggctgctcctggaccattatttgaaaatgtattgggtggtagaaatccgatcgatggactatggaattcagcccaaggtatgcttggtggctttggcggtggccttggaaaatga

SEQ ID NO: 60 - DNA, 171 bp, Artificial Sequence
  Synthetic
atgtggtcaatgtcttttgccttgcttttgatcgcttgtgtcattgccatggtcacggctgctcctggaccattatttgagaatgtattgggtggtagaaatccgatcgatggactatggaattcagcacaaggtatgcttggtggctttggcggtggccttggaaaatga

SEQ ID NO: 61 - DNA, 171 bp, Artificial Sequence
  Synthetic
atgtggtcaatgtcttttgccttgcgtttgatcgcttgtgtcattgccatggtcacggctgctcctggaccattatttgagaatgtattgggtggtagaaatccgatcgatggactatggaattcagcacaaggtatgcttggtggctttggcggtggccttggaaaatga

SEQ ID NO: 62 - DNA, 171 bp, Artificial Sequence
  Synthetic
atgtggtcaatgtctcttgccttgcttttgatcgcttgtgtcattgccatggtcacggctgctcctggaccattatttgagaatgtattgggtggtagaaatccgatcgatggactatggaattcagcacaaggtatgcttggtggctttggcggtggccttggaaaatga

SEQ ID NO: 63 - DNA, 171 bp, Artificial Sequence
  Synthetic
atgtggtcaatgtcttttgccttgcttttgaacgcttgtgtcattgccatggtcacggctgctcctggaccattatttgagaatgtattgggtggtagaaatccgatcgatggactatggaattcagcacaaggtatgcttggtggctttggcggtggccttggaaaatga

SEQ ID NO: 64 - DNA, 171 bp, Artificial Sequence
  Synthetic
atgtggtcaatgtcttttgccttgcttttgaccgcttgtgtcattgccatggtcacggctgctcctggaccattatttgagaatgtattgggtggcagaaatccgatcgatggactatggaattcagcacaaggtatgcttggtggctttggcggtggccttggaaaatga

SEQ ID NO: 65 - protein, 66 aa, Artificial Sequence
  Synthetic
MRSLSFALVLMAYAIAMVMAAPKPFFNLLSPLDGLLGGIDSVAHAGAQVLGLEPQQQYRQQGGPYY

SEQ ID NO: 66 - protein, 66 aa, Artificial Sequence
  Synthetic
MWSLTFSLILMACTIAMVMAAPKPFFNLLSPLDGLLGGIDSVAHAGAQVLGLEPQQQYRQQGGPYY

SEQ ID NO: 67 - protein, 66 aa, Artificial Sequence
  Synthetic
MWSLTFSLILMACTIAMVLAAPKPFFNLLSPLDGLLGGIDSVAHAGAQVLGLEPQQQYRQQGGYNY

SEQ ID NO: 68 - protein, 66 aa, Artificial Sequence
  Synthetic
MWSLTFSLILMACTIAMVMAAPKPFFNLLSPLDGLLGGIDSVAHAGAQVLGLEPQQQYRQRGGYNY

SEQ ID NO: 69 - protein, 66 aa, Artificial Sequence
  Synthetic
MWSLTFSLILMACTIAMVMAAPKPFFNLLSPFDGLLGGIDSVAHAGAQVLGLEPQQQYRQQGGYNY

SEQ ID NO: 70 - protein, 66 aa, Artificial Sequence
  Synthetic
MWSLTFSLILMACTIAMVMAAPKPFSNLLSPLDGLLGGIDSVAHAGAQVLGLEPQQQYRQQGGYNY

SEQ ID NO: 71 - protein, 66 aa, Artificial Sequence
  Synthetic
MWSLTFSLILMACTIAMVMAAPKPFFNLLSPLDGLLGGIDSVAHAGAQVLGLEPQQQYRQQGGYNY

SEQ ID NO: 72 - protein, 55 aa, Artificial Sequence
  Synthetic
MLLPWSWQLPNHFWGGVFPQFDSLLQGVDNVLHDGAQLIGLEPQQQYRQQGGPYY

SEQ ID NO: 73 - protein, 57 aa, Artificial Sequence
  Synthetic
MAYAIAMVMAAPKPFLGGVFPQFDSLLQGVDNVLHDGAQLIGLEPQQQYRQQGGPYY

SEQ ID NO: 74 - protein, 56 aa, Artificial Sequence
  Synthetic
MAYAIAMVMAAPKPFLGGVFPQFDSLLQGVDDVLHDGAQLIGLEPQQQYRQQGGPY

SEQ ID NO: 75 - protein, 67 aa, Artificial Sequence
  Synthetic
MRSLSFALAFDGYAIAMVMAAPKPFLGGVFPQFDSLLQGVDNVLHDGARLIGLEPQQQYRQQGGPYY

SEQ ID NO: 76 - protein, 67 aa, Artificial Sequence
  Synthetic
MRSLSFALVLMAYAIAMVMAAPKPFLGAVFPQFDSLLRGVDNVLHDGAQLIGLEPQQQYRQQGGPYY

SEQ ID NO: 77 - protein, 67 aa, Artificial Sequence
  Synthetic
MRSLSFALVLMAYAIAMVMAAPKPFLGGVFPQFDSSLQGVDNVLHDGAQLIGLEPQQQYRQQGGPYY

SEQ ID NO: 78 - protein, 67 aa, Artificial Sequence
  Synthetic
MRSLSFALVLMAYAIAMVMAAPKPFLGGVFPQFDSLLQGVDNVLHDGAQLIGLEPQQPYRQQGGPYY

SEQ ID NO: 79 - protein, 67 aa, Artificial Sequence
  Synthetic
MRSLSFALVLMAYAIAMVMAAPKPFLGGVFPQFDSLLQGVDNVLHDGAQLIGLEPQQQYRQQGGPCY

SEQ ID NO: 80 - protein, 67 aa, Artificial Sequence
  Synthetic
MRSLSFALVLMAYAIAMVMAAPKPFLGGVFPQFDSLLQGVDNELHDGAQLIGLEPQQQYRQQGGPYY

SEQ ID NO: 81 - protein, 67 aa, Artificial Sequence
  Synthetic
MRSLSFALVLMAYAIAMVMAAPKPFLGGVFPQFDSLLQGVDNVLHDGVQLIGLEPQQQYRQQGGPYY

SEQ ID NO: 82 - protein, 67 aa, Artificial Sequence
  Synthetic
MRSLSFALVLMAYAIAMVMAAPKPFLGGVFPQFDSLLQVVDNVLHDGAQLIGLEPQQQYRQQGGPYY

SEQ ID NO: 83 - protein, 67 aa, Artificial Sequence
  Synthetic
MRSLSFALVLMAYAIAMVMAAPKPFLGGVFPQFDSLLQGVDNVLHDGAQFIGLEPQQQYRQQGGPYY

SEQ ID NO: 84 - protein, 67 aa, Artificial Sequence
  Synthetic
MRSLSFALVLMAYAIAMVMAAPKPFLGGVFPQFDSLLQGVDNVLHDGAQLFGLEPQQQYRQQGGPYY

SEQ ID NO: 85 - protein, 67 aa, Artificial Sequence
  Synthetic
MRSLSFALVLMAYAIAMVMAAPKPFLGGVFPQFDSLLQGVDNVLHDGAQLIGLEPQRQYRQQGGPYY

SEQ ID NO: 86 - protein, 67 aa, Artificial Sequence
  Synthetic
MRSLSFALVLMAYAIAMVMAAPKPFLGGVFPQFDSLLQGVDNVLHDGAQLIGLEPQQQCRQQGGPYY

SEQ ID NO: 87 - protein, 67 aa, Artificial Sequence
  Synthetic
MRSLSFALVLMAYAIAMVMAAPKPFLGGVFPQFDSLLQGVDKVLHDGAQLIGLEPQQQYRQQGGPYY

SEQ ID NO: 88 - protein, 67 aa, Artificial Sequence
  Synthetic
MRSLSFALVLMAYAIAMVVAAPKPFLGGVFPQFDSLLQGVDNVLRDGAQLIGLEPQQQYRQQGGPYY

SEQ ID NO: 89 - protein, 67 aa, Artificial Sequence
  Synthetic
MRSLSFALVLMAYAIAMVMAAPKPFLGGVFPQFDSLLQGVDNVLRDGAQLIGLEPQQQYRQQGGPYY

SEQ ID NO: 90 - protein, 67 aa, Artificial Sequence
  Synthetic
MRSLSFALVLLAYAIAMVMAAPKPFLGGVFPQFDSLLQGVDNVLHDGAQLIGLEPQQQYRQQGGPYY

SEQ ID NO: 91 - protein, 67 aa, Artificial Sequence
  Synthetic
MRSLSFALVLMAYAIAMVMAAPKPFLGGVFPQFDSLLQGVNNVLHDGAQLIGLEPQQQYRQQGGPYY

SEQ ID NO: 92 - protein, 67 aa, Artificial Sequence
  Synthetic
MRSLSFALVLMAYAIAMVMAAPKPFLGGVFPQFDSLLQGVDNVLHDGAQLIGLEPQQQYRQQGGPYH

SEQ ID NO: 93 - protein, 67 aa, Artificial Sequence
  Synthetic
MRSLSFALFLMAYAIAMVMAAPKPFLGGVFPQFDSLLQGVDNVLHDGAQLIGLEPQQQYRQQGGPYY

SEQ ID NO: 94 - protein, 67 aa, Artificial Sequence
  Synthetic
MRSLSFALVLTAYAIAMVMAAPKPFLGGVFPQFDSLLQGVDNVLHDGAQLIGLEPQQQYRQQGGPYY

SEQ ID NO: 95 - protein, 67 aa, Artificial Sequence
  Synthetic
MRSLSFALVLMAYAIAMVMAAPKPFLGGVFPLFDSLLQGVDNVLHDGAQLIGLEPQQQYRQQGGPYY

SEQ ID NO: 96 - protein, 67 aa, Artificial Sequence
  Synthetic
MRSLSFALVLMAYAIAMVMAAPKPFLGGVFPQFDSLLQGVDNVLHAGAQLIGLEPQQQYRQQGGPYY

SEQ ID NO: 97 - protein, 67 aa, Artificial Sequence
  Synthetic
MRSLSFALVLMAYAIAMVTAAPKPFLGGVFPQFDSLLQGVDNVLHDGAQLIGLEPQQQYRQQGGPYY

SEQ ID NO: 98 - protein, 67 aa, Artificial Sequence
  Synthetic
MRSLSFALVLMAYAIAMAMAAPKPFLGGVFPQFDSLLQGVDNVLHDGAQLIGLEPQQQYRQQGGPYY

SEQ ID NO: 99 - protein, 67 aa, Artificial Sequence
  Synthetic
MRSLSFALVLMAYAIAMVMAAPKPFLGGVFPQFDSLLQGVDDVLHDGAQLIGLEPQQQYRQQGGPYY

SEQ ID NO: 100 - protein, 67 aa, Artificial Sequence
  Synthetic
MRSLSFALVLMAYAIAMVMAAPKPFLGGVFPQFDSLLQGVDNVLHDGAQLVGLEPQQQYRQQGGPYY

SEQ ID NO: 101 - protein, 67 aa, Artificial Sequence
  Synthetic
MRSLSFALVLMAYAIAMVMAAPKPFLGGVFPQFDSLLQGVDNVLHDGAQLIGLEPQQQYRQQGGPYY

SEQ ID NO: 102 - protein, 67 aa, Artificial Sequence
  Synthetic
MWSLTFSLILMACTIAMVMAAPKPFLGGVFPQFDSLLQGVDNVLHDGAQLIGLEPQQQYRQQGGPYY

SEQ ID NO: 103 - protein, 63 aa, Artificial Sequence
  Synthetic
MMWSLTFTLLLIACVIATVIAAPQPFLGMDRMLGGIPIVSDVMNAMGGGGRGGSFGLIPGILK

SEQ ID NO: 104 - protein, 63 aa, Artificial Sequence
  Synthetic
MMWSLTFALLLVTCVIATVIAAPQPFLGMDRVLGGIPIVSDVMNAMGGGGRGGSFGLIPGILK

SEQ ID NO: 105 - protein, 63 aa, Artificial Sequence
  Synthetic
MTWSLTFALLLVTCVIATVIAAPQPFLGMDRMLGGIPIVSDVMNAMGGGGRGGSFGLIPGILK

SEQ ID NO: 106 - protein, 63 aa, Artificial Sequence
  Synthetic
MMWSLTFALLLVTRVIATVIAAPQPFLGMDRMLGGIPIVSDVMNAMGGGGRGGSFGLIPGILK

SEQ ID NO: 107 - protein, 63 aa, Artificial Sequence
  Synthetic
MMWSLTFALLLVTCVIATVIAAPQPFLGMDRMLGGIPIVSDAMNAMGGGGRGGSFGLIPGILK

SEQ ID NO: 108 - protein, 63 aa, Artificial Sequence
  Synthetic
MMWSLTFALLLVTCVIATVIAAPQPSLGMDRMLGGIPIVSDVMNAMGGGGRGGSFGLIPGILK

SEQ ID NO: 109 - protein, 63 aa, Artificial Sequence
  Synthetic
MMWSLTFALLLVTCVIATVIAAPQPFLGMDRMLGGIPIVSDVMNAMGGGGRGRSFGLIPGILK

SEQ ID NO: 110 - protein, 63 aa, Artificial Sequence
  Synthetic
MMWSLTFALLLVTCVIATVIAAPQPFLGMDRMLGGIPIVSDVMNAMGGGGRGGSFGLIPGILK

SEQ ID NO: 111 - protein, 62 aa, Artificial Sequence
  Synthetic
MWSMSFALLLIACVIATVIAAPQPFLGMDRMLGGIPIVSDVMNAMGGGGRGGSFGLIPGILK

SEQ ID NO: 112 - protein, 58 aa, Artificial Sequence
  Synthetic
MWSMSFALLLIACVIAMVTAAPGPMLEGVLGRGNPIDGPWNSAQGVFGGLGGGLGLGK

SEQ ID NO: 113 - protein, 58 aa, Artificial Sequence
  Synthetic
MWSMSFASLLIACVIAMVTAAPGPMLEGVLGRGNPIDGLWNSAQGVFGGLGGGLGLGK

SEQ ID NO: 114 - protein, 58 aa, Artificial Sequence
  Synthetic
MWSMSFALLLIACVIAMVTAAPGPMLEGVLGRGNPIDGLWNSAQGVFGGLGGGLGLGK

SEQ ID NO: 115 - protein, 58 aa, Artificial Sequence; identical to SEQ ID NO: 114
  Synthetic

SEQ ID NO: 116 - protein, 58 aa, Artificial Sequence
  Synthetic
MWSMSFALLLIACVIAMVTAAPGPMLEGVLGRGNPIDGLWNSAQGVFGGLGGGLGLVK

SEQ ID NO: 117 - protein, 56 aa, Artificial Sequence
  Synthetic
VWSMSFALLLIACVIAMVTAAPGPLFEKVLGGRNPIDGLWNSAQGMLGGFGGGLGK

SEQ ID NO: 118 - protein, 56 aa, Artificial Sequence
  Synthetic
MWSMSFALLLIACVIAMATAAPGPLFENVLGGRNPIDGLWNSAQGMLGGFGGGLGK

SEQ ID NO: 119 - protein, 56 aa, Artificial Sequence
  Synthetic
MWSMSFALLLIACVIAMVTAAPGPLFETVLGGRNPIDGLWNSAQGMLGGFGGGLGK

SEQ ID NO: 120 - protein, 56 aa, Artificial Sequence
  Synthetic
MWSMSFALLLIACVIAMVTAAPGPLFENVLGGRNPIDGLWNLAQGMLGGFGGGLGK

SEQ ID NO: 121 - protein, 56 aa, Artificial Sequence
  Synthetic
MWSMSFALLLIACVIAMATAAPGPLFENALGGRNPIDGLWNSAQGMLGGFGGGLGR

SEQ ID NO: 122 - protein, 56 aa, Artificial Sequence
  Synthetic
LGSMSFALLLIACVIAMVTAAPGPLFENVLGGRNPIDGLWNSAQGMLGGFGGGLGK

SEQ ID NO: 123 - protein, 56 aa, Artificial Sequence
  Synthetic
MWSMSFALLLIACVIAMVTAAPGPLFENVLGGRNPIDGLWNSAQGMLGGFGGGLGK

SEQ ID NO: 124 - protein, 56 aa, Artificial Sequence
  Synthetic
MWSMSFALRLIACVIAMVTAAPGPLFENVLGGRNPIDGLWNSAQGMLGGFGGGLGK

SEQ ID NO: 125 - protein, 56 aa, Artificial Sequence
  Synthetic
MWSMSLALLLIACVIAMVTAAPGPLFENVLGGRNPIDGLWNSAQGMLGGFGGGLGK

SEQ ID NO: 126 - protein, 56 aa, Artificial Sequence
  Synthetic
MWSMSFALLLNACVIAMVTAAPGPLFENVLGGRNPIDGLWNSAQGMLGGFGGGLGK

SEQ ID NO: 127 - protein, 56 aa, Artificial Sequence
  Synthetic
MWSMSFALLLTACVIAMVTAAPGPLFENVLGGRNPIDGLWNSAQGMLGGFGGGLGK

SEQ ID NO: 128 - protein, 58 aa, Artificial Sequence
  Synthetic
MWSMSFALLLIACVIAMVTAAPGPLFENVLGGRNPVDGLWNSAQGVFGGLGGGLGLGK

SEQ ID NO: 129 - DNA, 141 bp, Artificial Sequence
  Synthetic
gctcccaaaccgtttttcaacttactcagtccgcttgatggtttattaggaggaatagatagtgtggcgcatgcaggagcacaagttcttggccttgaaccacagcagcagtacaggcaacaagggggctacaactactga

SEQ ID NO: 130 - DNA, 141 bp, Artificial Sequence
  Synthetic
gctcccaaaccgtttttcaacttactcagtccgcttgatggtttattaggaggaatagatagtgtggcgcatgcaggagcacaagttcttggccttgaaccacagcagcagtacaggcaacgagggggctacaactactga

SEQ ID NO: 131 - DNA, 141 bp, Artificial Sequence
  Synthetic
gctcccaaaccgtttttcaacttactcagtccgtttgatggtttattaggaggaatagatagtgtggcgcatgcaggagcacaagttcttggccttgaaccacagcagcagtacaggcaacaagggggctacaactactga

SEQ ID NO: 132 - DNA, 141 bp, Artificial Sequence
  Synthetic
gctcccaaaccgttttccaacttactcagtccgcttgatggtttattaggaggaatagatagtgtggcgcatgcaggagcacaagttcttggccttgaaccacagcagcagtacaggcaacaagggggctacaactactga

SEQ ID NO: 133 - DNA, 141 bp, Artificial Sequence; identical to SEQ ID NO: 129
  Synthetic

SEQ ID NO: 134 - DNA, 141 bp, Artificial Sequence
  Synthetic
gctcccaaaccgtttttcaacttactcagtccgcttgatggtttattaggaggaatagatagtgtggcgcatgcaggagcacaagttcttggccttgaaccacagcagcagtacaggcaacaagggggcccttactactaa

SEQ ID NO: 135 - DNA, 141 bp, Artificial Sequence; identical to SEQ ID NO: 134
  Synthetic

SEQ ID NO: 136 - DNA, 168 bp, Artificial Sequence; identical to SEQ ID NO: 8
  Synthetic

SEQ ID NO: 137 - DNA, 144 bp, Artificial Sequence
  Synthetic
gctcccaaaccatttttggggggagtttttccgcagtttgatagtttattacaaggagtagataatgtgttgcatgatggagcacaacttattggccttgaaccacagcagcagtacaggcaacaagggggcccttactactaa

SEQ ID NO: 138 - DNA, 144 bp, Artificial Sequence
  Synthetic
gctcccaaaccatttttggggggagtctttccgcagtttgatagtttattacaaggagtagatgatgtgttgcatgatggagcacaacttattggccttgaaccacagcagcagtacaggcaacaagggggcccttactactaa

SEQ ID NO: 139 - DNA, 144 bp, Artificial Sequence
  Synthetic
gctcccaaaccatttttggggggagtttttccgcagtttgatagtttattacaaggagtagataatgtgttgcatgatggagcacgacttattggccttgaaccacagcagcagtacaggcaacaagggggcccttactactaa

SEQ ID NO: 140 - DNA, 144 bp, Artificial Sequence
  Synthetic
gctcccaaaccatttttgggggcagtttttccgcagtttgatagtttattacgaggagtagataatgtgttgcatgatggagcacaacttattggccttgaaccacagcagcagtacaggcaacaagggggcccttactactaa

SEQ ID NO: 141 - DNA, 144 bp, Artificial Sequence
  Synthetic
gctcccaaaccatttttggggggagtttttccgcagtttgatagttcattacaaggagtagataatgtgttgcatgatggagcacaacttattggccttgaaccacagcagcagtacaggcaacaagggggcccttactactaa

SEQ ID NO: 142 - DNA, 144 bp, Artificial Sequence
  Synthetic
gctcccaaaccatttttggggggagtttttccgcagtttgatagtttattacaaggagtagataacgtgttgcatgatggagcacaacttattggccttgaaccacagcagccgtacaggcaacaagggggcccttactactaa

SEQ ID NO: 143 - DNA, 144 bp, Artificial Sequence
  Synthetic
gctcccaaaccatttttggggggagtttttccgcagtttgatagtttattacaaggagtagataatgtgttgcatgatggagcacaacttattggccttgaaccacagcagcagtacaggcaacaagggggcccttgctactaa

SEQ ID NO: 144 - DNA, 144 bp, Artificial Sequence
  Synthetic
gctcccaaaccatttttggggggagtttttccgcagtttgatagtttattacaaggagtagataatgagttgcatgatggagcacaacttattggccttgaaccacagcagcagtacaggcaacaagggggcccttactactaa

SEQ ID NO: 145 - DNA, 144 bp, Artificial Sequence
  Synthetic
gctcccaaaccatttttggggggagtttttccgcagtttgatagtttattacaaggagtagataatgtgttgcatgatggagtacaacttattggccttgaaccacagcagcagtacaggcaacaagggggcccttactactaa

SEQ ID NO: 146 - DNA, 144 bp, Artificial Sequence
  Synthetic
gctcccaaaccatttttggggggagtttttccgcagtttgatagtttattacaagtagtagataatgtgttgcatgatggagcacaacttattggccttgaaccacagcagcagtacaggcaacaagggggcccttactactaa

SEQ ID NO: 147 - DNA, 144 bp, Artificial Sequence
  Synthetic
gctcccaaaccatttttggggggagtttttccgcagtttgatagtttattacaaggagtagataatgtgttgcatgatggagcacaatttattggccttgaaccacagcagcagtacaggcaacaagggggcccttactactaa

SEQ ID NO: 148 - DNA, 144 bp, Artificial Sequence
  Synthetic
gctcccaaaccatttttggggggagtttttccgcagtttgatagtttattacaaggagtagataatgtgttgcatgatggagcacaactttttggccttgaaccacagcagcagtacaggcaacaagggggcccttactactaa

SEQ ID NO: 149 - DNA, 144 bp, Artificial Sequence
  Synthetic
gctcccaaaccatttttggggggagtttttccgcagtttgatagtttattacaaggagtagataatgtgttgcatgatggagcacaacttattggccttgaaccacagcggcagtacaggcaacaagggggcccttactactaa

SEQ ID NO: 150 - DNA, 144 bp, Artificial Sequence
  Synthetic
gctcccaaaccatttttggggggagtttttccgcagtttgatagtttattacaaggagtagataatgtgttgcatgatggagcacaacttattggccttgaaccacagcagcagtgcaggcaacaagggggcccttactactaa

SEQ ID NO: 151 - DNA, 144 bp, Artificial Sequence
  Synthetic
gctcccaaaccatttttggggggagtttttccgcagtttgatagtttattacaaggagtagataaagtgttgcatgatggagcacaacttattggccttgaaccacagcagcagtacaggcaacaagggggcccttactactaa

SEQ ID NO: 152 - DNA, 144 bp, Artificial Sequence
  Synthetic
gctcccaaaccatttttggggggagtttttccgcagtttgatagtttattacaaggagtagataatgtgttgcgtgatggagcacaacttattggccttgaaccacagcagcagtacaggcaacaagggggcccttactactaa

SEQ ID NO: 153 - DNA, 144 bp, Artificial Sequence; identical to SEQ ID NO: 152
  Synthetic

SEQ ID NO: 154 - DNA, 144 bp, Artificial Sequence; identical to SEQ ID NO: 137
  Synthetic

SEQ ID NO: 155 - DNA, 144 bp, Artificial Sequence
  Synthetic
gctcccaaaccatttttggggggagtttttccgcagtttgatagtttattacaaggagtaaataatgtgttgcatgatggagcacaacttattggccttgaaccacagcagcagtacaggcaacaagggggcccttactactaa

SEQ ID NO: 156 - DNA, 144 bp, Artificial Sequence
  Synthetic
gctcccaaaccatttttggggggagtttttccgcagtttgatagtttattacaaggagtagataatgtgttgcatgatggagcacaacttattggccttgaaccacagcagcagtacaggcaacaagggggcccttaccactaa

SEQ ID NO: 157 - DNA, 144 bp, Artificial Sequence; identical to SEQ ID NO: 137
  Synthetic

SEQ ID NO: 158 - DNA, 144 bp, Artificial Sequence
  Synthetic
gctcccaaaccatttttggggggagtgtttccgcagtttgatagtttattacaaggagtagataatgtgttgcatgatggagcacaacttattggccttgaaccacagcagcagtacaggcaacaagggggcccttactactaa

SEQ ID NO: 159 - DNA, 144 bp, Artificial Sequence
  Synthetic
gctcccaaaccatttttggggggagtttttccgctgtttgatagtttattacaaggagtagataatgtgttgcatgatggagcacaacttattggccttgaaccacagcagcagtacaggcaacaagggggcccttactactaa

SEQ ID NO: 160 - DNA, 144 bp, Artificial Sequence
  Synthetic
gctcccaaaccatttttggggggagtttttccgcagtttgatagtttattacaaggagtagataatgtgttgcatgcaggagcacaacttattggccttgaaccacagcagcagtacaggcaacaagggggcccttactactaa

SEQ ID NO: 161 - DNA, 144 bp, Artificial Sequence; identical to SEQ ID NO: 137
  Synthetic

SEQ ID NO: 162 - DNA, 144 bp, Artificial Sequence; identical to SEQ ID NO: 137
  Synthetic

SEQ ID NO: 163 - DNA, 144 bp, Artificial Sequence
  Synthetic
gctcccaaaccatttttggggggagtttttccgcagtttgatagtttattacaaggagtagatgatgtgttgcatgatggagcacaacttattggccttgaaccacagcagcagtacaggcaacaagggggcccttactactaa

SEQ ID NO: 164 - DNA, 144 bp, Artificial Sequence
  Synthetic
gctcccaaaccatttttggggggagtttttccgcagtttgatagtttattacaaggagtagataatgtgttgcatgatggagcacaacttgttggccttgaaccacagcagcagtacaggcaacaagggggcccttactactaa

SEQ ID NO: 165 - DNA, 144 bp, Artificial Sequence; identical to SEQ ID NO: 137
  Synthetic

SEQ ID NO: 166 - DNA, 144 bp, Artificial Sequence; identical to SEQ ID NO: 137
  Synthetic

SEQ ID NO: 167 - DNA, 129 bp, Artificial Sequence
  Synthetic
gctccccaaccctttcttggaatggatagaatgctaggtggtataccaattgtcagtgatgtgatgaatgcaatgggcggcggcggccgcggcggtagcttcgggctcatccccgggatcctaaaatag

SEQ ID NO: 168 - DNA, 129 bp, Artificial Sequence
  Synthetic
gctccccaaccctttcttggaatggatagagtgctaggtggtataccaattgtcagtgatgtgatgaatgcaatgggcggcggcggccgcggcggtagcttcgggctcatccccgggatcctaaaatag

SEQ ID NO: 169 - DNA, 129 bp, Artificial Sequence; identical to SEQ ID NO: 167
  Synthetic

SEQ ID NO: 170 - DNA, 129 bp, Artificial Sequence; identical to SEQ ID NO: 167
  Synthetic

SEQ ID NO: 171 - DNA, 129 bp, Artificial Sequence
  Synthetic
gctccccaaccctttcttggaatggatagaatgctaggtggtataccaattgtcagtgatgcgatgaatgcaatgggcggcggcggccgcggcggtagcttcgggctcatccccgggatcctaaaatag

SEQ ID NO: 172 - DNA, 129 bp, Artificial Sequence
  Synthetic
gctccccaaccctctcttggaatggatagaatgctaggtggtataccaatcgtcagtgatgtgatgaatgcaatgggcggcggcggccgcggcggtagcttcgggctcatccccgggatcctaaaatag

SEQ ID NO: 173 - DNA, 129 bp, Artificial Sequence
  Synthetic
gctccccaaccctttcttggaatggatagaatgctaggtggtataccaattgtcagtgatgtgatgaatgcaatgggcggcggcggccgcggccgtagcttcgggctcatccccgggatcctaaaatag

SEQ ID NO: 174 - DNA, 129 bp, Artificial Sequence; identical to SEQ ID NO: 167
  Synthetic

SEQ ID NO: 175 - DNA, 129 bp, Artificial Sequence; identical to SEQ ID NO: 167
  Synthetic

SEQ ID NO: 176 - DNA, 117 bp, Artificial Sequence
  Synthetic
gctcctggacctatgcttgagggtgtattgggtcgtggaaatccgatcgatggaccatggaattcagcacaaggtgtgtttggtggtttgggcggtggcctcggccttggaaaatga

SEQ ID NO: 177 - DNA, 117 bp, Artificial Sequence
  Synthetic
gctcctggacctatgcttgagggtgtattgggtcgtggaaatccgatcgatggactatggaattcagcacaaggtgtgtttggtggtttgggcggtggcctcggccttggaaaatga

SEQ ID NO: 178 - DNA, 117 bp, Artificial Sequence; identical to SEQ ID NO: 177
  Synthetic

SEQ ID NO: 179 - DNA, 117 bp, Artificial Sequence; identical to SEQ ID NO: 177
  Synthetic

SEQ ID NO: 180 - DNA, 117 bp, Artificial Sequence
  Synthetic
gctcctggacctatgcttgagggtgtattgggtcgtggaaatccgatcgatggactatggaattcagcacaaggtgtgtttggtggtttgggcggtggcctcggccttgtaaaatga

SEQ ID NO: 181 - DNA, 117 bp, Artificial Sequence
  Synthetic
gctcctggaccattatttgagaatgtattgggtggtagaaatccggtcgatggactatggaattcagcacaaggtgtgtttggtggtttgggcggtggcctcggccttggaaaatga

SEQ ID NO: 182 - DNA, 111 bp, Artificial Sequence
  Synthetic
gctcctggaccattatttgagaaagtattgggtggtagaaatccgatcgatggactatggaattcagcacaaggtatgcttggtggctttggcggtggccttggaaaatga

SEQ ID NO: 183 - DNA, 111 bp, Artificial Sequence
  Synthetic
gctcctggaccattatttgagaatgtattgggtggtagaaatccgatcgatggactatggaattcagcacaaggtatgcttggtggctttggcggtggccttggaaaatga

SEQ ID NO: 184 - DNA, 111 bp, Artificial Sequence
  Synthetic
gctcctggaccattatttgagactgtattgggtggtagaaatccgatcgatggactatggaattcagcacaaggtatgcttggtggctttggcggtggccttggaaaatga

SEQ ID NO: 185 - DNA, 111 bp, Artificial Sequence
  Synthetic
gctcctggaccattatttgagaatgtattgggtggtagaaatccgatcgatggactatggaatttagcacaaggtatgcttggtggctttggcggtggccttggaaaatga

SEQ ID NO: 186 - DNA, 111 bp, Artificial Sequence
  Synthetic
gctcctggaccattatttgagaatgcattgggtggtagaaatccgatcgatggactatggaattcagcacaaggtatgcttggtggctttggcggtggccttggaagatga

SEQ ID NO: 187 - DNA, 111 bp, Artificial Sequence
  Synthetic
gctcctggaccattatttgaaaatgtattgggtggtagaaatccgatcgatggactatggaattcagcccaaggtatgcttggtggctttggcggtggccttggaaaatga

SEQ ID NO: 188 - DNA, 111 bp, Artificial Sequence; identical to SEQ ID NO: 183
  Synthetic

SEQ ID NO: 189 - DNA, 111 bp, Artificial Sequence; identical to SEQ ID NO: 183
  Synthetic

SEQ ID NO: 190 - DNA, 111 bp, Artificial Sequence; identical to SEQ ID NO: 183
  Synthetic

SEQ ID NO: 191 - DNA, 111 bp, Artificial Sequence; identical to SEQ ID NO: 183
  Synthetic

SEQ ID NO: 192 - DNA, 111 bp, Artificial Sequence
  Synthetic
gctcctggaccattatttgagaatgtattgggtggcagaaatccgatcgatggactatggaattcagcacaaggtatgcttggtggctttggcggtggccttggaaaatga

SEQ ID NO: 193 - protein, 46 aa, Artificial Sequence
  Synthetic
APKPFFNLLSPLDGLLGGIDSVAHAGAQVLGLEPQQQYRQQGGPYY

SEQ ID NO: 194 - protein, 46 aa, Artificial Sequence; identical to SEQ ID NO: 193
  Synthetic

SEQ ID NO: 195 - protein, 46 aa, Artificial Sequence
  Synthetic
APKPFFNLLSPLDGLLGGIDSVAHAGAQVLGLEPQQQYRQQGGYNY

SEQ ID NO: 196 - protein, 46 aa, Artificial Sequence
  Synthetic
APKPFFNLLSPLDGLLGGIDSVAHAGAQVLGLEPQQQYRQRGGYNY

SEQ ID NO: 197 - protein, 46 aa, Artificial Sequence
  Synthetic
APKPFFNLLSPFDGLLGGIDSVAHAGAQVLGLEPQQQYRQQGGYNY

SEQ ID NO: 198 - protein, 46 aa, Artificial Sequence
  Synthetic
APKPFSNLLSPLDGLLGGIDSVAHAGAQVLGLEPQQQYRQQGGYNY

SEQ ID NO: 199 - protein, 46 aa, Artificial Sequence; identical to SEQ ID NO: 195
  Synthetic

SEQ ID NO: 200 - protein, 55 aa, Artificial Sequence; identical to SEQ ID NO: 72
  Synthetic

SEQ ID NO: 201 - protein, 47 aa, Artificial Sequence
  Synthetic
APKPFLGGVFPQFDSLLQGVDNVLHDGAQLIGLEPQQQYRQQGGPYY

SEQ ID NO: 202 - protein, 46 aa, Artificial Sequence
  Synthetic
APKPFLGGVFPQFDSLLQGVDDVLHDGAQLIGLEPQQQYRQQGGPY

SEQ ID NO: 203 - protein, 47 aa, Artificial Sequence
  Synthetic
APKPFLGGVFPQFDSLLQGVDNVLHDGARLIGLEPQQQYRQQGGPYY

SEQ ID NO: 204 - protein, 47 aa, Artificial Sequence
  Synthetic
APKPFLGAVFPQFDSLLRGVDNVLHDGAQLIGLEPQQQYRQQGGPYY

SEQ ID NO: 205 - protein, 47 aa, Artificial Sequence
  Synthetic
APKPFLGGVFPQFDSSLQGVDNVLHDGAQLIGLEPQQQYRQQGGPYY

SEQ ID NO: 206 - protein, 47 aa, Artificial Sequence
  Synthetic
APKPFLGGVFPQFDSLLQGVDNVLHDGAQLIGLEPQQPYRQQGGPYY

SEQ ID NO: 207 - protein, 47 aa, Artificial Sequence
  Synthetic
APKPFLGGVFPQFDSLLQGVDNVLHDGAQLIGLEPQQQYRQQGGPCY

SEQ ID NO: 208 - protein, 47 aa, Artificial Sequence
  Synthetic
APKPFLGGVFPQFDSLLQGVDNELHDGAQLIGLEPQQQYRQQGGPYY

SEQ ID NO: 209 - protein, 47 aa, Artificial Sequence
  Synthetic
APKPFLGGVFPQFDSLLQGVDNVLHDGVQLIGLEPQQQYRQQGGPYY

SEQ ID NO: 210 - protein, 47 aa, Artificial Sequence
  Synthetic
APKPFLGGVFPQFDSLLQVVDNVLHDGAQLIGLEPQQQYRQQGGPYY

SEQ ID NO: 211 - protein, 47 aa, Artificial Sequence
  Synthetic
APKPFLGGVFPQFDSLLQGVDNVLHDGAQFIGLEPQQQYRQQGGPYY

SEQ ID NO: 212 - protein, 47 aa, Artificial Sequence
  Synthetic
APKPFLGGVFPQFDSLLQGVDNVLHDGAQLFGLEPQQQYRQQGGPYY

SEQ ID NO: 213 - protein, 47 aa, Artificial Sequence
  Synthetic
APKPFLGGVFPQFDSLLQGVDNVLHDGAQLIGLEPQRQYRQQGGPYY

SEQ ID NO: 214 - protein, 47 aa, Artificial Sequence
  Synthetic
APKPFLGGVFPQFDSLLQGVDNVLHDGAQLIGLEPQQQCRQQGGPYY

SEQ ID NO: 215 - protein, 47 aa, Artificial Sequence
  Synthetic
APKPFLGGVFPQFDSLLQGVDKVLHDGAQLIGLEPQQQYRQQGGPYY

SEQ ID NO: 216 - protein, 47 aa, Artificial Sequence
  Synthetic
APKPFLGGVFPQFDSLLQGVDNVLRDGAQLIGLEPQQQYRQQGGPYY

SEQ ID NO: 217 - protein, 47 aa, Artificial Sequence; identical to SEQ ID NO: 216
  Synthetic

SEQ ID NO: 218 - protein, 47 aa, Artificial Sequence; identical to SEQ ID NO: 201
  Synthetic

SEQ ID NO: 219 - protein, 47 aa, Artificial Sequence
  Synthetic
APKPFLGGVFPQFDSLLQGVNNVLHDGAQLIGLEPQQQYRQQGGPYY

SEQ ID NO: 220 - protein, 47 aa, Artificial Sequence
  Synthetic
APKPFLGGVFPQFDSLLQGVDNVLHDGAQLIGLEPQQQYRQQGGPYH

SEQ ID NO: 221 - protein, 47 aa, Artificial Sequence; identical to SEQ ID NO: 201
  Synthetic

SEQ ID NO: 222 - protein, 47 aa, Artificial Sequence; identical to SEQ ID NO: 201
  Synthetic

SEQ ID NO: 223 - protein, 47 aa, Artificial Sequence
  Synthetic
APKPFLGGVFPLFDSLLQGVDNVLHDGAQLIGLEPQQQYRQQGGPYY

SEQ ID NO: 224 - protein, 47 aa, Artificial Sequence
  Synthetic
APKPFLGGVFPQFDSLLQGVDNVLHAGAQLIGLEPQQQYRQQGGPYY

SEQ ID NO: 225 - protein, 47 aa, Artificial Sequence; identical to SEQ ID NO: 201
  Synthetic

SEQ ID NO: 226 - protein, 47 aa, Artificial Sequence; identical to SEQ ID NO: 201
  Synthetic

SEQ ID NO: 227 - protein, 47 aa, Artificial Sequence
  Synthetic
APKPFLGGVFPQFDSLLQGVDDVLHDGAQLIGLEPQQQYRQQGGPYY

SEQ ID NO: 228 - protein, 47 aa, Artificial Sequence
  Synthetic
APKPFLGGVFPQFDSLLQGVDNVLHDGAQLVGLEPQQQYRQQGGPYY

SEQ ID NO: 229 - protein, 47 aa, Artificial Sequence; identical to SEQ ID NO: 201
  Synthetic

SEQ ID NO: 230 - protein, 47 aa, Artificial Sequence; identical to SEQ ID NO: 201
  Synthetic

SEQ ID NO: 231 - protein, 42 aa, Artificial Sequence
  Synthetic
APQPFLGMDRMLGGIPIVSDVMNAMGGGGRGGSFGLIPGILK

SEQ ID NO: 232 - protein, 42 aa, Artificial Sequence
  Synthetic
APQPFLGMDRVLGGIPIVSDVMNAMGGGGRGGSFGLIPGILK

SEQ ID NO: 233 - protein, 42 aa, Artificial Sequence; identical to SEQ ID NO: 231
  Synthetic

SEQ ID NO: 234 - protein, 42 aa, Artificial Sequence; identical to SEQ ID NO: 231
  Synthetic

SEQ ID NO: 235 - protein, 42 aa, Artificial Sequence
  Synthetic
APQPFLGMDRMLGGIPIVSDAMNAMGGGGRGGSFGLIPGILK

SEQ ID NO: 236 - protein, 42 aa, Artificial Sequence
  Synthetic
APQPSLGMDRMLGGIPIVSDVMNAMGGGGRGGSFGLIPGILK

SEQ ID NO: 237 - protein, 42 aa, Artificial Sequence
  Synthetic
APQPFLGMDRMLGGIPIVSDVMNAMGGGGRGRSFGLIPGILK

SEQ ID NO: 238 - protein, 42 aa, Artificial Sequence; identical to SEQ ID NO: 231
  Synthetic

SEQ ID NO: 239 - protein, 42 aa, Artificial Sequence; identical to SEQ ID NO: 231
  Synthetic

SEQ ID NO: 240 - protein, 38 aa, Artificial Sequence
  Synthetic
APGPMLEGVLGRGNPIDGPWNSAQGVFGGLGGGLGLGK

SEQ ID NO: 241 - protein, 38 aa, Artificial Sequence
  Synthetic
APGPMLEGVLGRGNPIDGLWNSAQGVFGGLGGGLGLGK

SEQ ID NO: 242 - protein, 38 aa, Artificial Sequence; identical to SEQ ID NO: 241
  Synthetic

SEQ ID NO: 243 - protein, 38 aa, Artificial Sequence; identical to SEQ ID NO: 241
  Synthetic

SEQ ID NO: 244 - protein, 38 aa, Artificial Sequence
  Synthetic
APGPMLEGVLGRGNPIDGLWNSAQGVFGGLGGGLGLVK

SEQ ID NO: 245 - protein, 36 aa, Artificial Sequence
  Synthetic
APGPLFEKVLGGRNPIDGLWNSAQGMLGGFGGGLGK

SEQ ID NO: 246 - protein, 36 aa, Artificial Sequence
  Synthetic
APGPLFENVLGGRNPIDGLWNSAQGMLGGFGGGLGK

SEQ ID NO: 247 - protein, 36 aa, Artificial Sequence
  Synthetic
APGPLFETVLGGRNPIDGLWNSAQGMLGGFGGGLGK

SEQ ID NO: 248 - protein, 36 aa, Artificial Sequence
  Synthetic
APGPLFENVLGGRNPIDGLWNLAQGMLGGFGGGLGK

SEQ ID NO: 249 - protein, 36 aa, Artificial Sequence
  Synthetic
APGPLFENALGGRNPIDGLWNSAQGMLGGFGGGLGR

SEQ ID NO: 250 - protein, 36 aa, Artificial Sequence; identical to SEQ ID NO: 246
  Synthetic

SEQ ID NO: 251 - protein, 36 aa, Artificial Sequence; identical to SEQ ID NO: 246
  Synthetic

SEQ ID NO: 252 - protein, 36 aa, Artificial Sequence; identical to SEQ ID NO: 246
  Synthetic

SEQ ID NO: 253 - protein, 36 aa, Artificial Sequence; identical to SEQ ID NO: 246
  Synthetic

SEQ ID NO: 254 - protein, 36 aa, Artificial Sequence; identical to SEQ ID NO: 246
  Synthetic

SEQ ID NO: 255 - protein, 36 aa, Artificial Sequence; identical to SEQ ID NO: 246
  Synthetic

SEQ ID NO: 256 - protein, 38 aa, Artificial Sequence
  Synthetic
APGPLFENVLGGRNPVDGLWNSAQGVFGGLGGGLGLGK

SEQ ID NO: 257 - DNA, 186 bp, Artificial Sequence
  Synthetic
atgtggacaatgtcctttgccttgcttttggtcgcttgtgtcgttgccatggtcacggctgctcctggaccttttctaggtctttttgagccacagcaagcttataatggttatcctcaaaatggtggaattgctcaagatatagctagcgtttttcatggcttgagcggcggcatccttcgataa

SEQ ID NO: 258 - DNA, 186 bp, Artificial Sequence
  Synthetic
atgtggacaatgtcccttgccttgcttttggtcgcttgtgtcgttgccatggtcacggctgctcctggaccttttctaggtctttttgagccacagcaagcttataatggttatcctcaaaatggtggaattgctcaagatatagctagcgtttttcatggcttgagcggcggcatccttcgataa

SEQ ID NO: 259 - DNA, 186 bp, Artificial Sequence
  Synthetic
atgtggacaatgtcctttgccttgctttttgtcgcttgtgtcgttgccatggtcacggctgctccaggaccttttctaggtctttttgagccacagcaagcttataatggttatcctcaaaatggtggaattgctcaagatatagctggtatggttcatggcttgagcggcggcatccttcgataa

SEQ ID NO: 260 - DNA, 204 bp, Artificial Sequence
  Synthetic
atgcggtctctgtcttttgccctagttttgatggcttatgctattgccatggtcatggcagctcccaaaccatttttggggggagtttttccgcagtttgatagtttattacaaggagtanataatgtgttgcatgatggagcacaacttattggccttgaaccacagcagcagtacaggcaacaagggggcccttactactaa

SEQ ID NO: 261 - protein, 61 aa, Artificial Sequence
  Synthetic
MWTMSFALLLVACVVAMVTAAPGPFLGLFEPQQAYNGYPQNGGIAQDIASVFHGLSGGILR

SEQ ID NO: 262 - protein, 61 aa, Artificial Sequence
  Synthetic
MWTMSLALLLVACVVAMVTAAPGPFLGLFEPQQAYNGYPQNGGIAQDIASVFHGLSGGILR

SEQ ID NO: 263 - protein, 61 aa, Artificial Sequence
  Synthetic
MWTMSFALLFVACVVAMVTAAPGPFLGLFEPQQAYNGYPQNGGIAQDIAGMVHGLSGGILR

SEQ ID NO: 264 - protein, 67 aa, Artificial Sequence
  Synthetic
MRSLSFALVLMAYAIAMVMAAPKPFLGGVFPQFDSLLQGVXNVLHDGAQLIGLEPQQQYRQQGGPYY

SEQ ID NO: 265 - DNA, 126 bp, Artificial Sequence
  Synthetic
gctcctggaccttttctaggtctttttgagccacagcaagcttataatggttatcctcaaaatggtggaattgctcaagatatagctagcgtttttcatggcttgagcggcggcatccttcgataa

SEQ ID NO: 266 - DNA, 126 bp, Artificial Sequence; identical to SEQ ID NO: 265
  Synthetic

SEQ ID NO: 267 - DNA, 126 bp, Artificial Sequence
  Synthetic
gctccaggaccttttctaggtctttttgagccacagcaagcttataatggttatcctcaaaatggtggaattgctcaagatatagctggtatggttcatggcttgagcggcggcatccttcgataa

SEQ ID NO: 268 - DNA, 144 bp, Artificial Sequence
  Synthetic
gctcccaaaccatttttggggggagtttttccgcagtttgatagtttattacaaggagtanataatgtgttgcatgatggagcacaacttattggccttgaaccacagcagcagtacaggcaacaagggggcccttactactaa

SEQ ID NO: 269 - protein, 41 aa, Artificial Sequence
  Synthetic
APGPFLGLFEPQQAYNGYPQNGGIAQDIASVFHGLSGGILR

SEQ ID NO: 270 - protein, 41 aa, Artificial Sequence; identical to SEQ ID NO: 269
  Synthetic

SEQ ID NO: 271 - protein, 41 aa, Artificial Sequence
  Synthetic
APGPFLGLFEPQQAYNGYPQNGGIAQDIAGMVHGLSGGILR

SEQ ID NO: 272 - protein, 47 aa, Artificial Sequence
  Synthetic
APKPFLGGVFPQFDSLLQGVXNVLHDGAQLIGLEPQQQYRQQGGPYY

SEQ ID NO: 273 - protein, 4 aa, Artificial Sequence
  Synthetic
APXP

SEQ ID NO: 274 - protein, 21 aa, Artificial Sequence
  Synthetic
GAXXXGLEPQQQYRQQGGPYY

SEQ ID NO: 275 - protein, 22 aa, Artificial Sequence
  Synthetic
NPIDGPWNSAQGXXGGXGGGLG

SEQ ID NO: 276 - protein, 22 aa, Artificial Sequence
  Synthetic
NPIDGLWNSAQGXXGGXGGGLG